SEQUENCE LISTING (312 sequences)

SEQ ID NO: 1 - DNA, 82 bp, synthetic construct
  misc_feature 1..82: note = Synthetic Construct
  source 1..82: mol_type = other DNA
acccacgtccctggcggaggaaataaaaaggtgcagataattaataagaagctggatcttagcaacgtccagtccaagtgtg

SEQ ID NO: 2 - DNA, 21 bp, synthetic construct
  misc_feature 1..21: note = Synthetic Construct
  variation 20..21
  source 1..21: mol_type = other DNA
ggtgcagataattaataagnn

SEQ ID NO: 3 - DNA, 21 bp, synthetic construct
  misc_feature 1..21: note = Synthetic Construct
  variation 20..21
  source 1..21: mol_type = other DNA
aggtgcagataattaataann

SEQ ID NO: 4 - DNA, 21 bp, synthetic construct
  misc_feature 1..21: note = Synthetic Construct
  misc_difference 20..21: note = n is a, c, g, t, or u
  source 1..21: mol_type = other DNA
aaggtgcagataattaatann

SEQ ID NO: 5 - DNA, 21 bp, synthetic construct
  misc_feature 1..21: note = Synthetic Construct
  variation 20..21
  source 1..21: mol_type = other DNA
aaaggtgcagataattaatnn

SEQ ID NO: 6 - DNA, 21 bp, synthetic construct
  misc_feature 1..21: note = Synthetic Construct
  variation 20..21
  source 1..21: mol_type = other DNA
aaaaggtgcagataattaann

SEQ ID NO: 7 - DNA, 21 bp, synthetic construct
  misc_feature 1..21: note = Synthetic Construct
  variation 20..21
  source 1..21: mol_type = other DNA
aaaaaggtgcagataattann

SEQ ID NO: 8 - DNA, 21 bp, synthetic construct
  misc_feature 1..21: note = Synthetic Construct
  variation 20..21
  source 1..21: mol_type = other DNA
taaaaaggtgcagataattnn

SEQ ID NO: 9 - DNA, 21 bp, synthetic construct
  misc_feature 1..21: note = Synthetic Construct
  variation 20..21
  source 1..21: mol_type = other DNA
ataaaaaggtgcagataatnn

SEQ ID NO: 10 - DNA, 21 bp, synthetic construct
  misc_feature 1..21: note = Synthetic Construct
  variation 20..21
  source 1..21: mol_type = other DNA
aataaaaaggtgcagataann

SEQ ID NO: 11 - DNA, 21 bp, synthetic construct
  misc_feature 1..21: note = Synthetic Construct
  variation 20..21
  source 1..21: mol_type = other DNA
aaataaaaaggtgcagatann

SEQ ID NO: 12 - DNA, 21 bp, synthetic construct
  misc_feature 1..21: note = Synthetic Construct
  variation 20..21
  source 1..21: mol_type = other DNA
gaaataaaaaggtgcagatnn

SEQ ID NO: 13 - DNA, 21 bp, synthetic construct
  misc_feature 1..21: note = Synthetic Construct
  variation 20..21
  source 1..21: mol_type = other DNA
ggaaataaaaaggtgcagann

SEQ ID NO: 14 - DNA, 21 bp, synthetic construct
  misc_feature 1..21: note = Synthetic Construct
  variation 20..21
  source 1..21: mol_type = other DNA
aggaaataaaaaggtgcagnn

SEQ ID NO: 15 - DNA, 21 bp, synthetic construct
  misc_feature 1..21: note = Synthetic Construct
  variation 20..21
  source 1..21: mol_type = other DNA
gaggaaataaaaaggtgcann

SEQ ID NO: 16 - DNA, 21 bp, synthetic construct
  misc_feature 1..21: note = Synthetic Construct
  variation 20..21
  source 1..21: mol_type = other DNA
ggaggaaataaaaaggtgcnn

SEQ ID NO: 17 - DNA, 21 bp, synthetic construct
  misc_feature 1..21: note = Synthetic Construct
  variation 20..21
  source 1..21: mol_type = other DNA
cggaggaaataaaaaggtgnn

SEQ ID NO: 18 - DNA, 21 bp, synthetic construct
  misc_feature 1..21: note = Synthetic Construct
  variation 20..21
  source 1..21: mol_type = other DNA
gcggaggaaataaaaaggtnn

SEQ ID NO: 19 - DNA, 21 bp, synthetic construct
  misc_feature 1..21: note = Synthetic Construct
  variation 20..21
  source 1..21: mol_type = other DNA
ggcggaggaaataaaaaggnn

SEQ ID NO: 20 - DNA, 21 bp, synthetic construct
  misc_feature 1..21: note = Synthetic Construct
  variation 20..21
  source 1..21: mol_type = other DNA
cttattaattatctgcaccnn

SEQ ID NO: 21 - DNA, 21 bp, synthetic construct
  misc_feature 1..21: note = Synthetic Construct
  variation 20..21
  source 1..21: mol_type = other DNA
ttattaattatctgcacctnn

SEQ ID NO: 22 - DNA, 21 bp, synthetic construct
  misc_feature 1..21: note = Synthetic Construct
  variation 20..21
  source 1..21: mol_type = other DNA
tattaattatctgcaccttnn

SEQ ID NO: 23 - DNA, 21 bp, synthetic construct
  misc_feature 1..21: note = Synthetic Construct
  variation 20..21
  source 1..21: mol_type = other DNA
attaattatctgcacctttnn

SEQ ID NO: 24 - DNA, 21 bp, synthetic construct
  misc_feature 1..21: note = Synthetic Construct
  variation 20..21
  source 1..21: mol_type = other DNA
ttaattatctgcaccttttnn

SEQ ID NO: 25 - DNA, 21 bp, synthetic construct
  misc_feature 1..21: note = Synthetic Construct
  variation 20..21
  source 1..21: mol_type = other DNA
taattatctgcacctttttnn

SEQ ID NO: 26 - DNA, 21 bp, synthetic construct
  misc_feature 1..21: note = Synthetic Construct
  variation 20..21
  source 1..21: mol_type = other DNA
aattatctgcacctttttann

SEQ ID NO: 27 - DNA, 21 bp, synthetic construct
  misc_feature 1..21: note = Synthetic Construct
  misc_feature 20..21: note = n is a, c, g, t or u
  source 1..21: mol_type = other DNA
attatctgcacctttttatnn

SEQ ID NO: 28 - DNA, 21 bp, synthetic construct
  misc_feature 1..21: note = Synthetic Construct
  variation 20..21
  source 1..21: mol_type = other DNA
ttatctgcacctttttattnn

SEQ ID NO: 29 - DNA, 21 bp, synthetic construct
  misc_feature 1..21: note = Synthetic Construct
  variation 20..21
  source 1..21: mol_type = other DNA
tatctgcacctttttatttnn

SEQ ID NO: 30 - DNA, 21 bp, synthetic construct
  misc_feature 1..21: note = Synthetic Construct
  variation 20..21
  source 1..21: mol_type = other DNA
atctgcacctttttatttcnn

SEQ ID NO: 31 - DNA, 21 bp, synthetic construct
  misc_feature 1..21: note = Synthetic Construct
  variation 20..21
  source 1..21: mol_type = other DNA
tctgcacctttttatttccnn

SEQ ID NO: 32 - DNA, 21 bp, synthetic construct
  misc_feature 1..21: note = Synthetic Construct
  variation 20..21
  source 1..21: mol_type = other DNA
ctgcacctttttatttcctnn

SEQ ID NO: 33 - DNA, 21 bp, synthetic construct
  misc_feature 1..21: note = Synthetic Construct
  variation 20..21
  source 1..21: mol_type = other DNA
tgcacctttttatttcctcnn

SEQ ID NO: 34 - DNA, 21 bp, synthetic construct
  misc_feature 1..21: note = Synthetic Construct
  variation 20..21
  source 1..21: mol_type = other DNA
gcacctttttatttcctccnn

SEQ ID NO: 35 - DNA, 21 bp, synthetic construct
  misc_feature 1..21: note = Synthetic Construct
  variation 20..21
  source 1..21: mol_type = other DNA
cacctttttatttcctccgnn

SEQ ID NO: 36 - DNA, 21 bp, synthetic construct
  misc_feature 1..21: note = Synthetic Construct
  variation 20..21
  source 1..21: mol_type = other DNA
acctttttatttcctccgcnn

SEQ ID NO: 37 - DNA, 21 bp, synthetic construct
  misc_feature 1..21: note = Synthetic Construct
  variation 20..21
  source 1..21: mol_type = other DNA
cctttttatttcctccgccnn

SEQ ID NO: 38 - skipped

SEQ ID NO: 39 - DNA, 86 bp, synthetic construct
  misc_feature 1..86: note = Synthetic Construct
  source 1..86: mol_type = other DNA
acccacgtccctggcggaggaaataaaaagggggctgatggtaaaacgaagatcgccacaccgcggggagcagcccctccaggcca

SEQ ID NO: 40 - DNA, 21 bp, synthetic construct
  misc_feature 1..21: note = Synthetic Construct
  variation 20..21
  source 1..21: mol_type = other DNA
gggggctgatggtaaaacgnn

SEQ ID NO: 41 - DNA, 21 bp, synthetic construct
  misc_feature 1..21: note = Synthetic Construct
  variation 20..21
  source 1..21: mol_type = other DNA
agggggctgatggtaaaacnn

SEQ ID NO: 42 - DNA, 21 bp, synthetic construct
  misc_feature 1..21: note = Synthetic Construct
  variation 20..21
  source 1..21: mol_type = other DNA
aagggggctgatggtaaaann

SEQ ID NO: 43 - DNA, 21 bp, synthetic construct
  misc_feature 1..21: note = Synthetic Construct
  variation 20..21
  source 1..21: mol_type = other DNA
aaagggggctgatggtaaann

SEQ ID NO: 44 - DNA, 21 bp, synthetic construct
  misc_feature 1..21: note = Synthetic Construct
  variation 20..21
  source 1..21: mol_type = other DNA
aaaagggggctgatggtaann

SEQ ID NO: 45 - DNA, 21 bp, synthetic construct
  misc_feature 1..21: note = Synthetic Construct
  variation 20..21
  source 1..21: mol_type = other DNA
aaaaagggggctgatggtann

SEQ ID NO: 46 - DNA, 21 bp, synthetic construct
  misc_feature 1..21: note = Synthetic Construct
  variation 20..21
  source 1..21: mol_type = other DNA
taaaaagggggctgatggtnn

SEQ ID NO: 47 - DNA, 21 bp, synthetic construct
  misc_feature 1..21: note = Synthetic Construct
  variation 20..21
  source 1..21: mol_type = other DNA
ataaaaagggggctgatggnn

SEQ ID NO: 48 - DNA, 21 bp, synthetic construct
  misc_feature 1..21: note = Synthetic Construct
  variation 20..21
  source 1..21: mol_type = other DNA
aataaaaagggggctgatgnn

SEQ ID NO: 49 - DNA, 21 bp, synthetic construct
  misc_feature 1..21: note = Synthetic Construct
  variation 20..21
  source 1..21: mol_type = other DNA
aaataaaaagggggctgatnn

SEQ ID NO: 50 - DNA, 21 bp, synthetic construct
  misc_feature 1..21: note = Synthetic Construct
  variation 20..21
  source 1..21: mol_type = other DNA
gaaataaaaagggggctgann

SEQ ID NO: 51 - DNA, 21 bp, synthetic construct
  misc_feature 1..21: note = Synthetic Construct
  variation 20..21
  source 1..21: mol_type = other DNA
ggaaataaaaagggggctgnn

SEQ ID NO: 52 - DNA, 21 bp, synthetic construct
  misc_feature 1..21: note = Synthetic Construct
  variation 20..21
  source 1..21: mol_type = other DNA
aggaaataaaaagggggctnn

SEQ ID NO: 53 - DNA, 21 bp, synthetic construct
  misc_feature 1..21: note = Synthetic Construct
  variation 20..21
  source 1..21: mol_type = other DNA
gaggaaataaaaagggggcnn

SEQ ID NO: 54 - DNA, 21 bp, synthetic construct
  misc_feature 1..21: note = Synthetic Construct
  variation 20..21
  source 1..21: mol_type = other DNA
ggaggaaataaaaagggggnn

SEQ ID NO: 55 - DNA, 21 bp, synthetic construct
  misc_feature 1..21: note = Synthetic Construct
  variation 20..21
  source 1..21: mol_type = other DNA
cggaggaaataaaaaggggnn

SEQ ID NO: 56 - DNA, 21 bp, synthetic construct
  misc_feature 1..21: note = Synthetic Construct
  variation 20..21
  source 1..21: mol_type = other DNA
gcggaggaaataaaaagggnn

SEQ ID NO: 57 - DNA, 21 bp, synthetic construct
  misc_feature 1..21: note = Synthetic Construct
  variation 20..21
  source 1..21: mol_type = other DNA
ggcggaggaaataaaaaggnn

SEQ ID NO: 58 - DNA, 21 bp, synthetic construct
  misc_feature 1..21: note = Synthetic Construct
  variation 20..21
  source 1..21: mol_type = other DNA
cgttttaccatcagcccccnn

SEQ ID NO: 59 - DNA, 21 bp, synthetic construct
  misc_feature 1..21: note = Synthetic Construct
  variation 20..21
  source 1..21: mol_type = other DNA
gttttaccatcagccccctnn

SEQ ID NO: 60 - DNA, 21 bp, synthetic construct
  misc_feature 1..21: note = Synthetic Construct
  variation 20..21
  source 1..21: mol_type = other DNA
ttttaccatcagcccccttnn

SEQ ID NO: 61 - DNA, 21 bp, synthetic construct
  misc_feature 1..21: note = Synthetic Construct
  variation 20..21
  source 1..21: mol_type = other DNA
tttaccatcagccccctttnn

SEQ ID NO: 62 - DNA, 21 bp, synthetic construct
  misc_feature 1..21: note = Synthetic Construct
  variation 20..21
  source 1..21: mol_type = other DNA
ttaccatcagcccccttttnn

SEQ ID NO: 63 - DNA, 21 bp, synthetic construct
  misc_feature 1..21: note = Synthetic Construct
  variation 20..21
  source 1..21: mol_type = other DNA
taccatcagccccctttttnn

SEQ ID NO: 64 - DNA, 21 bp, synthetic construct
  misc_feature 1..21: note = Synthetic Construct
  variation 20..21
  source 1..21: mol_type = other DNA
accatcagccccctttttann

SEQ ID NO: 65 - DNA, 21 bp, synthetic construct
  misc_feature 1..21: note = Synthetic Construct
  variation 20..21
  source 1..21: mol_type = other DNA
ccatcagccccctttttatnn

SEQ ID NO: 66 - DNA, 21 bp, synthetic construct
  misc_feature 1..21: note = Synthetic Construct
  variation 20..21
  source 1..21: mol_type = other DNA
catcagccccctttttattnn

SEQ ID NO: 67 - DNA, 21 bp, synthetic construct
  misc_feature 1..21: note = Synthetic Construct
  variation 20..21
  source 1..21: mol_type = other DNA
atcagccccctttttatttnn

SEQ ID NO: 68 - DNA, 21 bp, synthetic construct
  misc_feature 1..21: note = Synthetic Construct
  variation 20..21
  source 1..21: mol_type = other DNA
tcagccccctttttatttcnn

SEQ ID NO: 69 - DNA, 21 bp, synthetic construct
  misc_feature 1..21: note = Synthetic Construct
  variation 20..21
  source 1..21: mol_type = other DNA
cagccccctttttatttccnn

SEQ ID NO: 70 - DNA, 21 bp, synthetic construct
  misc_feature 1..21: note = Synthetic Construct
  variation 20..21
  source 1..21: mol_type = other DNA
agccccctttttatttcctnn

SEQ ID NO: 71 - DNA, 21 bp, synthetic construct
  misc_feature 1..21: note = Synthetic Construct
  variation 20..21
  source 1..21: mol_type = other DNA
gccccctttttatttcctcnn

SEQ ID NO: 72 - DNA, 21 bp, synthetic construct
  misc_feature 1..21: note = Synthetic Construct
  variation 20..21
  source 1..21: mol_type = other DNA
ccccctttttatttcctccnn

SEQ ID NO: 73 - DNA, 21 bp, synthetic construct
  misc_feature 1..21: note = Synthetic Construct
  variation 20..21
  source 1..21: mol_type = other DNA
cccctttttatttcctccgnn

SEQ ID NO: 74 - DNA, 21 bp, synthetic construct
  misc_feature 1..21: note = Synthetic Construct
  variation 20..21
  source 1..21: mol_type = other DNA
ccctttttatttcctccgcnn

SEQ ID NO: 75 - DNA, 21 bp, synthetic construct
  misc_feature 1..21: note = Synthetic Construct
  variation 20..21
  source 1..21: mol_type = other DNA
cctttttatttcctccgccnn

SEQ ID NO: 76 - DNA, 40 bp, synthetic construct
  misc_feature 1..40: note = Synthetic Construct
  source 1..40: mol_type = other DNA
ctggcggaggaaataaaaaggtgcagataattaataagaa

SEQ ID NO: 77 - DNA, 40 bp, synthetic construct
  misc_feature 1..40: note = Synthetic Construct
  source 1..40: mol_type = other DNA
ctggcggaggaaataaaaagggggctgatggtaaaacgaa

SEQ ID NO: 78 - skipped

SEQ ID NO: 79 - DNA, 82 bp, synthetic construct; identical to SEQ ID NO: 1
  misc_feature 1..82: note = Synthetic Construct
  source 1..82: mol_type = other DNA

SEQ ID NO: 80 - DNA, 40 bp, synthetic construct
  source 1..40: mol_type = other DNA
tcattcccataacgacccagggccagctctcaatgttgca

SEQ ID NO: 81 - DNA, 22 bp, synthetic construct
  source 1..22: mol_type = other DNA
gggccagctctcaatgttgcnn

SEQ ID NO: 82 - DNA, 22 bp, synthetic construct
  source 1..22: mol_type = other DNA
agggccagctctcaatgttgnn

SEQ ID NO: 83 - DNA, 22 bp, synthetic construct
  source 1..22: mol_type = other DNA
cagggccagctctcaatgttnn

SEQ ID NO: 84 - DNA, 22 bp, synthetic construct
  source 1..22: mol_type = other DNA
ccagggccagctctcaatgtnn

SEQ ID NO: 85 - DNA, 22 bp, synthetic construct
  source 1..22: mol_type = other DNA
cccagggccagctctcaatgnn

SEQ ID NO: 86 - DNA, 22 bp, synthetic construct
  source 1..22: mol_type = other DNA
acccagggccagctctcaatnn

SEQ ID NO: 87 - DNA, 22 bp, synthetic construct
  source 1..22: mol_type = other DNA
gacccagggccagctctcaann

SEQ ID NO: 88 - DNA, 22 bp, synthetic construct
  source 1..22: mol_type = other DNA
cgacccagggccagctctcann

SEQ ID NO: 89 - DNA, 22 bp, synthetic construct
  source 1..22: mol_type = other DNA
acgacccagggccagctctcnn

SEQ ID NO: 90 - DNA, 22 bp, synthetic construct
  source 1..22: mol_type = other DNA
aacgacccagggccagctctnn

SEQ ID NO: 91 - DNA, 22 bp, synthetic construct
  source 1..22: mol_type = other DNA
taacgacccagggccagctcnn

SEQ ID NO: 92 - DNA, 22 bp, synthetic construct
  source 1..22: mol_type = other DNA
ataacgacccagggccagctnn

SEQ ID NO: 93 - DNA, 22 bp, synthetic construct
  source 1..22: mol_type = other DNA
cataacgacccagggccagcnn

SEQ ID NO: 94 - DNA, 22 bp, synthetic construct
  source 1..22: mol_type = other DNA
ccataacgacccagggccagnn

SEQ ID NO: 95 - DNA, 22 bp, synthetic construct
  source 1..22: mol_type = other DNA
cccataacgacccagggccann

SEQ ID NO: 96 - DNA, 22 bp, synthetic construct
  source 1..22: mol_type = other DNA
tcccataacgacccagggccnn

SEQ ID NO: 97 - DNA, 22 bp, synthetic construct
  source 1..22: mol_type = other DNA
ttcccataacgacccagggcnn

SEQ ID NO: 98 - DNA, 22 bp, synthetic construct
  source 1..22: mol_type = other DNA
attcccataacgacccagggnn

SEQ ID NO: 99 - DNA, 22 bp, synthetic construct
  source 1..22: mol_type = other DNA
cattcccataacgacccaggnn

SEQ ID NO: 100 - DNA, 40 bp, synthetic construct
  source 1..40: mol_type = other DNA
aagaaggatgctgttaaaagggccagctctcaatgttgca

SEQ ID NO: 101 - DNA, 22 bp, synthetic construct
  source 1..22: mol_type = other DNA
gggccagctctcaatgttgcnn

SEQ ID NO: 102 - DNA, 22 bp, synthetic construct
  source 1..22: mol_type = other DNA
agggccagctctcaatgttgnn

SEQ ID NO: 103 - DNA, 22 bp, synthetic construct
  source 1..22: mol_type = other DNA
aagggccagctctcaatgttnn

SEQ ID NO: 104 - DNA, 22 bp, synthetic construct
  source 1..22: mol_type = other DNA
aaagggccagctctcaatgtnn

SEQ ID NO: 105 - DNA, 22 bp, synthetic construct
  source 1..22: mol_type = other DNA
aaaagggccagctctcaatgnn

SEQ ID NO: 106 - DNA, 22 bp, synthetic construct
  source 1..22: mol_type = other DNA
taaaagggccagctctcaatnn

SEQ ID NO: 107 - DNA, 22 bp, synthetic construct
  source 1..22: mol_type = other DNA
ttaaaagggccagctctcaann

SEQ ID NO: 108 - DNA, 22 bp, synthetic construct
  source 1..22: mol_type = other DNA
gttaaaagggccagctctcann

SEQ ID NO: 109 - DNA, 22 bp, synthetic construct
  source 1..22: mol_type = other DNA
tgttaaaagggccagctctcnn

SEQ ID NO: 110 - DNA, 22 bp, synthetic construct
  source 1..22: mol_type = other DNA
ctgttaaaagggccagctctnn

SEQ ID NO: 111 - DNA, 22 bp, synthetic construct
  source 1..22: mol_type = other DNA
gctgttaaaagggccagctcnn

SEQ ID NO: 112 - DNA, 22 bp, synthetic construct
  source 1..22: mol_type = other DNA
tgctgttaaaagggccagctnn

SEQ ID NO: 113 - DNA, 22 bp, synthetic construct
  source 1..22: mol_type = other DNA
atgctgttaaaagggccagcnn

SEQ ID NO: 114 - DNA, 22 bp, synthetic construct
  source 1..22: mol_type = other DNA
gatgctgttaaaagggccagnn

SEQ ID NO: 115 - DNA, 22 bp, synthetic construct
  source 1..22: mol_type = other DNA
ggatgctgttaaaagggccann

SEQ ID NO: 116 - DNA, 22 bp, synthetic construct
  source 1..22: mol_type = other DNA
aggatgctgttaaaagggccnn

SEQ ID NO: 117 - DNA, 22 bp, synthetic construct
  source 1..22: mol_type = other DNA
aaggatgctgttaaaagggcnn

SEQ ID NO: 118 - DNA, 22 bp, synthetic construct
  source 1..22: mol_type = other DNA
gaaggatgctgttaaaagggnn

SEQ ID NO: 119 - DNA, 22 bp, synthetic construct
  source 1..22: mol_type = other DNA
agaaggatgctgttaaaaggnn

SEQ ID NO: 120 - DNA, 40 bp, synthetic construct
  source 1..40: mol_type = other DNA
aggctgagtgcggagccaagatacagtttggaactttatc

SEQ ID NO: 121 - DNA, 22 bp, synthetic construct
  source 1..22: mol_type = other DNA
gatacagtttggaactttatnn

SEQ ID NO: 122 - DNA, 22 bp, synthetic construct
  source 1..22: mol_type = other DNA
agatacagtttggaactttann

SEQ ID NO: 123 - DNA, 22 bp, synthetic construct
  source 1..22: mol_type = other DNA
aagatacagtttggaactttnn

SEQ ID NO: 124 - DNA, 22 bp, synthetic construct
  source 1..22: mol_type = other DNA
caagatacagtttggaacttnn

SEQ ID NO: 125 - DNA, 22 bp, synthetic construct
  source 1..22: mol_type = other DNA
ccaagatacagtttggaactnn

SEQ ID NO: 126 - DNA, 22 bp, synthetic construct
  source 1..22: mol_type = other DNA
gccaagatacagtttggaacnn

SEQ ID NO: 127 - DNA, 22 bp, synthetic construct
  source 1..22: mol_type = other DNA
agccaagatacagtttggaann

SEQ ID NO: 128 - DNA, 22 bp, synthetic construct
  source 1..22: mol_type = other DNA
gagccaagatacagtttggann

SEQ ID NO: 129 - DNA, 22 bp, synthetic construct
  source 1..22: mol_type = other DNA
ggagccaagatacagtttggnn

SEQ ID NO: 130 - DNA, 22 bp, synthetic construct
  source 1..22: mol_type = other DNA
cggagccaagatacagtttgnn

SEQ ID NO: 131 - DNA, 22 bp, synthetic construct
  source 1..22: mol_type = other DNA
gcggagccaagatacagtttnn

SEQ ID NO: 132 - DNA, 22 bp, synthetic construct
  source 1..22: mol_type = other DNA
tgcggagccaagatacagttnn

SEQ ID NO: 133 - DNA, 22 bp, synthetic construct
  source 1..22: mol_type = other DNA
gtgcggagccaagatacagtnn

SEQ ID NO: 134 - DNA, 22 bp, synthetic construct
  source 1..22: mol_type = other DNA
agtgcggagccaagatacagnn

SEQ ID NO: 135 - DNA, 22 bp, synthetic construct
  source 1..22: mol_type = other DNA
gagtgcggagccaagatacann

SEQ ID NO: 136 - DNA, 22 bp, synthetic construct
  source 1..22: mol_type = other DNA
tgagtgcggagccaagatacnn

SEQ ID NO: 137 - DNA, 22 bp, synthetic construct
  source 1..22: mol_type = other DNA
ctgagtgcggagccaagatann

SEQ ID NO: 138 - DNA, 22 bp, synthetic construct
  source 1..22: mol_type = other DNA
gctgagtgcggagccaagatnn

SEQ ID NO: 139 - DNA, 22 bp, synthetic construct
  source 1..22: mol_type = other DNA
ggctgagtgcggagccaagann

SEQ ID NO: 140 - DNA, 40 bp, synthetic construct
  source 1..40: mol_type = other DNA
ccgactataccacatacagggtaccttgtggtctataatc

SEQ ID NO: 141 - DNA, 22 bp, synthetic construct
  source 1..22: mol_type = other DNA
ggtaccttgtggtctataatnn

SEQ ID NO: 142 - DNA, 22 bp, synthetic construct
  source 1..22: mol_type = other DNA
gggtaccttgtggtctataann

SEQ ID NO: 143 - DNA, 22 bp, synthetic construct
  source 1..22: mol_type = other DNA
agggtaccttgtggtctatann

SEQ ID NO: 144 - DNA, 22 bp, synthetic construct
  source 1..22: mol_type = other DNA
cagggtaccttgtggtctatnn

SEQ ID NO: 145 - DNA, 22 bp, synthetic construct
  source 1..22: mol_type = other DNA
acagggtaccttgtggtctann

SEQ ID NO: 146 - DNA, 22 bp, synthetic construct
  source 1..22: mol_type = other DNA
tacagggtaccttgtggtctnn

SEQ ID NO: 147 - DNA, 22 bp, synthetic construct
  source 1..22: mol_type = other DNA
atacagggtaccttgtggtcnn

SEQ ID NO: 148 - DNA, 22 bp, synthetic construct
  source 1..22: mol_type = other DNA
catacagggtaccttgtggtnn

SEQ ID NO: 149 - DNA, 22 bp, synthetic construct
  source 1..22: mol_type = other DNA
acatacagggtaccttgtggnn

SEQ ID NO: 150 - DNA, 22 bp, synthetic construct
  source 1..22: mol_type = other DNA
cacatacagggtaccttgtgnn

SEQ ID NO: 151 - DNA, 22 bp, synthetic construct
  source 1..22: mol_type = other DNA
ccacatacagggtaccttgtnn

SEQ ID NO: 152 - DNA, 22 bp, synthetic construct
  source 1..22: mol_type = other DNA
accacatacagggtaccttgnn

SEQ ID NO: 153 - DNA, 22 bp, synthetic construct
  source 1..22: mol_type = other DNA
taccacatacagggtaccttnn

SEQ ID NO: 154 - DNA, 22 bp, synthetic construct
  source 1..22: mol_type = other DNA
ataccacatacagggtacctnn

SEQ ID NO: 155 - DNA, 22 bp, synthetic construct
  source 1..22: mol_type = other DNA
tataccacatacagggtaccnn

SEQ ID NO: 156 - DNA, 22 bp, synthetic construct
  source 1..22: mol_type = other DNA
ctataccacatacagggtacnn

SEQ ID NO: 157 - DNA, 22 bp, synthetic construct
  source 1..22: mol_type = other DNA
actataccacatacagggtann

SEQ ID NO: 158 - DNA, 22 bp, synthetic construct
  source 1..22: mol_type = other DNA
gactataccacatacagggtnn

SEQ ID NO: 159 - DNA, 22 bp, synthetic construct
  source 1..22: mol_type = other DNA
cgactataccacatacagggnn

SEQ ID NO: 160 - DNA, 40 bp, synthetic construct
  source 1..40: mol_type = other DNA
aaaattgattatggaaacagctttttataactatgatgcc

SEQ ID NO: 161 - DNA, 22 bp, synthetic construct
  source 1..22: mol_type = other DNA
gctttttataactatgatgcnn

SEQ ID NO: 162 - DNA, 22 bp, synthetic construct
  source 1..22: mol_type = other DNA
agctttttataactatgatgnn

SEQ ID NO: 163 - DNA, 22 bp, synthetic construct
  source 1..22: mol_type = other DNA
cagctttttataactatgatnn

SEQ ID NO: 164 - DNA, 22 bp, synthetic construct
  source 1..22: mol_type = other DNA
acagctttttataactatgann

SEQ ID NO: 165 - DNA, 22 bp, synthetic construct
  source 1..22: mol_type = other DNA
aacagctttttataactatgnn

SEQ ID NO: 166 - DNA, 22 bp, synthetic construct
  source 1..22: mol_type = other DNA
aaacagctttttataactatnn

SEQ ID NO: 167 - DNA, 22 bp, synthetic construct
  source 1..22: mol_type = other DNA
gaaacagctttttataactann

SEQ ID NO: 168 - DNA, 22 bp, synthetic construct
  source 1..22: mol_type = other DNA
ggaaacagctttttataactnn

SEQ ID NO: 169 - DNA, 22 bp, synthetic construct
  source 1..22: mol_type = other DNA
tggaaacagctttttataacnn

SEQ ID NO: 170 - DNA, 22 bp, synthetic construct
  source 1..22: mol_type = other DNA
atggaaacagctttttataann

SEQ ID NO: 171 - DNA, 22 bp, synthetic construct
  source 1..22: mol_type = other DNA
tatggaaacagctttttatann

SEQ ID NO: 172 - DNA, 22 bp, synthetic construct
  source 1..22: mol_type = other DNA
ttatggaaacagctttttatnn

SEQ ID NO: 173 - DNA, 22 bp, synthetic construct
  source 1..22: mol_type = other DNA
attatggaaacagctttttann

SEQ ID NO: 174 - DNA, 22 bp, synthetic construct
  source 1..22: mol_type = other DNA
gattatggaaacagctttttnn

SEQ ID NO: 175 - DNA, 22 bp, synthetic construct
  source 1..22: mol_type = other DNA
tgattatggaaacagcttttnn

SEQ ID NO: 176 - DNA, 22 bp, synthetic construct
  source 1..22: mol_type = other DNA
ttgattatggaaacagctttnn

SEQ ID NO: 177 - DNA, 22 bp, synthetic construct
  source 1..22: mol_type = other DNA
attgattatggaaacagcttnn

SEQ ID NO: 178 - DNA, 22 bp, synthetic construct
  source 1..22: mol_type = other DNA
aattgattatggaaacagctnn

SEQ ID NO: 179 - DNA, 22 bp, synthetic construct
  source 1..22: mol_type = other DNA
aaattgattatggaaacagcnn

SEQ ID NO: 180 - DNA, 40 bp, synthetic construct
  source 1..40: mol_type = other DNA
gcgaccttttggagtggctgctttttataactatgatgcc

SEQ ID NO: 181 - DNA, 22 bp, synthetic construct
  source 1..22: mol_type = other DNA
gctttttataactatgatgcnn

SEQ ID NO: 182 - DNA, 22 bp, synthetic construct
  source 1..22: mol_type = other DNA
tgctttttataactatgatgnn

SEQ ID NO: 183 - DNA, 22 bp, synthetic construct
  source 1..22: mol_type = other DNA
ctgctttttataactatgatnn

SEQ ID NO: 184 - DNA, 22 bp, synthetic construct
  source 1..22: mol_type = other DNA
gctgctttttataactatgann

SEQ ID NO: 185 - DNA, 22 bp, synthetic construct
  source 1..22: mol_type = other DNA
ggctgctttttataactatgnn

SEQ ID NO: 186 - DNA, 22 bp, synthetic construct
  source 1..22: mol_type = other DNA
tggctgctttttataactatnn

SEQ ID NO: 187 - DNA, 22 bp, synthetic construct
  source 1..22: mol_type = other DNA
gtggctgctttttataactann

SEQ ID NO: 188 - DNA, 22 bp, synthetic construct
  source 1..22: mol_type = other DNA
agtggctgctttttataactnn

SEQ ID NO: 189 - DNA, 22 bp, synthetic construct
  source 1..22: mol_type = other DNA
gagtggctgctttttataacnn

SEQ ID NO: 190 - DNA, 22 bp, synthetic construct
  source 1..22: mol_type = other DNA
ggagtggctgctttttataann

SEQ ID NO: 191 - DNA, 22 bp, synthetic construct
  source 1..22: mol_type = other DNA
tggagtggctgctttttatann

SEQ ID NO: 192 - DNA, 22 bp, synthetic construct
  source 1..22: mol_type = other DNA
ttggagtggctgctttttatnn

SEQ ID NO: 193 - DNA, 22 bp, synthetic construct
  source 1..22: mol_type = other DNA
tttggagtggctgctttttann

SEQ ID NO: 194 - DNA, 22 bp, synthetic construct
  source 1..22: mol_type = other DNA
ttttggagtggctgctttttnn

SEQ ID NO: 195 - DNA, 22 bp, synthetic construct
  source 1..22: mol_type = other DNA
cttttggagtggctgcttttnn

SEQ ID NO: 196 - DNA, 22 bp, synthetic construct
  source 1..22: mol_type = other DNA
ccttttggagtggctgctttnn

SEQ ID NO: 197 - DNA, 22 bp, synthetic construct
  source 1..22: mol_type = other DNA
accttttggagtggctgcttnn

SEQ ID NO: 198 - DNA, 22 bp, synthetic construct
  source 1..22: mol_type = other DNA
gaccttttggagtggctgctnn

SEQ ID NO: 199 - DNA, 22 bp, synthetic construct
  source 1..22: mol_type = other DNA
cgaccttttggagtggctgcnn

SEQ ID NO: 200 - DNA, 40 bp, synthetic construct
  source 1..40: mol_type = other DNA
gcattgccattttcacatagggaacaacctatcttcagca

SEQ ID NO: 201 - DNA, 22 bp, synthetic construct
  source 1..22: mol_type = other DNA
gggaacaacctatcttcagcnn

SEQ ID NO: 202 - DNA, 22 bp, synthetic construct
  source 1..22: mol_type = other DNA
agggaacaacctatcttcagnn

SEQ ID NO: 203 - DNA, 22 bp, synthetic construct
  source 1..22: mol_type = other DNA
tagggaacaacctatcttcann

SEQ ID NO: 204 - DNA, 22 bp, synthetic construct
  source 1..22: mol_type = other DNA
atagggaacaacctatcttcnn

SEQ ID NO: 205 - DNA, 22 bp, synthetic construct
  source 1..22: mol_type = other DNA
catagggaacaacctatcttnn

SEQ ID NO: 206 - DNA, 22 bp, synthetic construct
  source 1..22: mol_type = other DNA
acatagggaacaacctatctnn

SEQ ID NO: 207 - DNA, 22 bp, synthetic construct
  source 1..22: mol_type = other DNA
cacatagggaacaacctatcnn

SEQ ID NO: 208 - DNA, 22 bp, synthetic construct
  source 1..22: mol_type = other DNA
tcacatagggaacaacctatnn

SEQ ID NO: 209 - DNA, 22 bp, synthetic construct
  source 1..22: mol_type = other DNA
ttcacatagggaacaacctann

SEQ ID NO: 210 - DNA, 22 bp, synthetic construct
  source 1..22: mol_type = other DNA
tttcacatagggaacaacctnn

SEQ ID NO: 211 - DNA, 22 bp, synthetic construct
  source 1..22: mol_type = other DNA
ttttcacatagggaacaaccnn

SEQ ID NO: 212 - DNA, 22 bp, synthetic construct
  source 1..22: mol_type = other DNA
attttcacatagggaacaacnn

SEQ ID NO: 213 - DNA, 22 bp, synthetic construct
  source 1..22: mol_type = other DNA
cattttcacatagggaacaann

SEQ ID NO: 214 - DNA, 22 bp, synthetic construct
  source 1..22: mol_type = other DNA
ccattttcacatagggaacann

SEQ ID NO: 215 - DNA, 22 bp, synthetic construct
  source 1..22: mol_type = other DNA
gccattttcacatagggaacnn

SEQ ID NO: 216 - DNA, 22 bp, synthetic construct
  source 1..22: mol_type = other DNA
tgccattttcacatagggaann

SEQ ID NO: 217 - DNA, 22 bp, synthetic construct
  source 1..22: mol_type = other DNA
ttgccattttcacatagggann

SEQ ID NO: 218 - DNA, 22 bp, synthetic construct
  source 1..22: mol_type = other DNA
attgccattttcacatagggnn

SEQ ID NO: 219 - DNA, 22 bp, synthetic construct
  source 1..22: mol_type = other DNA
cattgccattttcacataggnn

SEQ ID NO: 220 - DNA, 40 bp, synthetic construct
  source 1..40: mol_type = other DNA
acatccagcttccacagatgcttatctggatgtcctctca

SEQ ID NO: 221 - DNA, 22 bp, synthetic construct
  source 1..22: mol_type = other DNA
gcttatctggatgtcctctcnn

SEQ ID NO: 222 - DNA, 22 bp, synthetic construct
  source 1..22: mol_type = other DNA
tgcttatctggatgtcctctnn

SEQ ID NO: 223 - DNA, 22 bp, synthetic construct
  source 1..22: mol_type = other DNA
atgcttatctggatgtcctcnn

SEQ ID NO: 224 - DNA, 22 bp, synthetic construct
  source 1..22: mol_type = other DNA
gatgcttatctggatgtcctnn

SEQ ID NO: 225 - DNA, 22 bp, synthetic construct
  source 1..22: mol_type = other DNA
agatgcttatctggatgtccnn

SEQ ID NO: 226 - DNA, 22 bp, synthetic construct
  source 1..22: mol_type = other DNA
cagatgcttatctggatgtcnn

SEQ ID NO: 227 - DNA, 22 bp, synthetic construct
  source 1..22: mol_type = other DNA
acagatgcttatctggatgtnn

SEQ ID NO: 228 - DNA, 22 bp, synthetic construct
  source 1..22: mol_type = other DNA
cacagatgcttatctggatgnn

SEQ ID NO: 229 - DNA, 22 bp, synthetic construct
  source 1..22: mol_type = other DNA
ccacagatgcttatctggatnn

SEQ ID NO: 230 - DNA, 22 bp, synthetic construct
  source 1..22: mol_type = other DNA
tccacagatgcttatctggann

SEQ ID NO: 231 - DNA, 22 bp, synthetic construct
  source 1..22: mol_type = other DNA
ttccacagatgcttatctggnn

SEQ ID NO: 232 - DNA, 22 bp, synthetic construct
  source 1..22: mol_type = other DNA
cttccacagatgcttatctgnn

SEQ ID NO: 233 - DNA, 22 bp, synthetic construct
  source 1..22: mol_type = other DNA
gcttccacagatgcttatctnn

SEQ ID NO: 234 - DNA, 22 bp, synthetic construct
  source 1..22: mol_type = other DNA
agcttccacagatgcttatcnn

SEQ ID NO: 235 - DNA, 22 bp, synthetic construct
  source 1..22: mol_type = other DNA
cagcttccacagatgcttatnn

SEQ ID NO: 236 - DNA, 22 bp, synthetic construct
  source 1..22: mol_type = other DNA
ccagcttccacagatgcttann

SEQ ID NO: 237 - DNA, 22 bp, synthetic construct
  source 1..22: mol_type = other DNA
tccagcttccacagatgcttnn

SEQ ID NO: 238 - DNA, 22 bp, synthetic construct
  source 1..22: mol_type = other DNA
atccagcttccacagatgctnn

SEQ ID NO: 239 - DNA, 22 bp, synthetic construct
  source 1..22: mol_type = other DNA
catccagcttccacagatgcnn

SEQ ID NO: 240 - DNA, 40 bp, synthetic construct
  source 1..40: mol_type = other DNA
atttagttgattctctgaagttgttgacctcctgtactgg

SEQ ID NO: 241 - DNA, 22 bp, synthetic construct
  source 1..22: mol_type = other DNA
gttgttgacctcctgtactgnn

SEQ ID NO: 242 - DNA, 22 bp, synthetic construct
  source 1..22: mol_type = other DNA
agttgttgacctcctgtactnn

SEQ ID NO: 243 - DNA, 22 bp, synthetic construct
  source 1..22: mol_type = other DNA
aagttgttgacctcctgtacnn

SEQ ID NO: 244 - DNA, 22 bp, synthetic construct
  source 1..22: mol_type = other DNA
gaagttgttgacctcctgtann

SEQ ID NO: 245 - DNA, 22 bp, synthetic construct
  source 1..22: mol_type = other DNA
tgaagttgttgacctcctgtnn

SEQ ID NO: 246 - DNA, 22 bp, synthetic construct
  source 1..22: mol_type = other DNA
ctgaagttgttgacctcctgnn

SEQ ID NO: 247 - DNA, 22 bp, synthetic construct
  source 1..22: mol_type = other DNA
tctgaagttgttgacctcctnn

SEQ ID NO: 248 - DNA, 22 bp, synthetic construct
  source 1..22: mol_type = other DNA
ctctgaagttgttgacctccnn

SEQ ID NO: 249 - DNA, 22 bp, synthetic construct
  source 1..22: mol_type = other DNA
tctctgaagttgttgacctcnn

SEQ ID NO: 250 - DNA, 22 bp, synthetic construct
  source 1..22: mol_type = other DNA
ttctctgaagttgttgacctnn

SEQ ID NO: 251 - DNA, 22 bp, synthetic construct
  source 1..22: mol_type = other DNA
attctctgaagttgttgaccnn

SEQ ID NO: 252 - DNA, 22 bp, synthetic construct
  source 1..22: mol_type = other DNA
gattctctgaagttgttgacnn

SEQ ID NO: 253 - DNA, 22 bp, synthetic construct
  source 1..22: mol_type = other DNA
tgattctctgaagttgttgann

SEQ ID NO: 254 - DNA, 22 bp, synthetic construct
  source 1..22: mol_type = other DNA
ttgattctctgaagttgttgnn

SEQ ID NO: 255 - DNA, 22 bp, synthetic construct
  source 1..22: mol_type = other DNA
gttgattctctgaagttgttnn

SEQ ID NO: 256 - DNA, 22 bp, synthetic construct
  source 1..22: mol_type = other DNA
agttgattctctgaagttgtnn

SEQ ID NO: 257 - DNA, 22 bp, synthetic construct
  source 1..22: mol_type = other DNA
tagttgattctctgaagttgnn

SEQ ID NO: 258 - DNA, 22 bp, synthetic construct
  source 1..22: mol_type = other DNA
ttagttgattctctgaagttnn

SEQ ID NO: 259 - DNA, 22 bp, synthetic construct
  source 1..22: mol_type = other DNA
tttagttgattctctgaagtnn

SEQ ID NO: 260 - protein, 13 aa, synthetic construct
IPITTQGQLSMLQ

SEQ ID NO: 261 - protein, 13 aa, synthetic construct
MRSSQILETQSSI

SEQ ID NO: 262 - protein, 34 aa, synthetic construct
VEFGSKDLTLLMNLMNGTLNPFKSLWCLIPITTQ

SEQ ID NO: 263 - protein, 54 aa, synthetic construct
MVRKVHKAISAKVLAHIFCPHNYPSQLTLQTVCLLHKVEVTIQMCRWWSLEARI

SEQ ID NO: 264 - protein, 116 aa, synthetic construct
MLQEKIDHLERLLAENNEIISNIRDSVINLSESVEDGPKSSQSNFSQGAGSHLLPSQLSLSVDTADCLFASQSGSHNSDVQMVEFGSKDLTLLMNLMNGTLNPFKSLWCLIPITTQ

SEQ ID NO: 265 - protein, 236 aa, synthetic construct
MLQEKIDHLERLLAENNEIISNIRDSVINLSESVEDGPKSSQSNFSQGAGSHLLPSQLSLSVDTADCLFASQSGSHNSDVQMVEFGSKDLTLLMNLMNGTLNPFKSLWCLIPITTQGQLSMLQEKIDHLERLLAENNEIISNIRDSVINLSESVEDGPKSSQSNFSQGAGSHLLPSQLSLSVDTADCLFASQSGSHNSDVQMVEFGSKDLTLLMNLMNGTLNPFKSLWCLIPITTQ

SEQ ID NO: 266 - protein, 18 aa, synthetic construct
GPALNVARKNRPFGAFAS

SEQ ID NO: 267 - protein, 52 aa, synthetic construct
MVRKVHKAISAKVLAHIFCPHNYPSQLTLQTVCLLHKVEVTIQMCRCWMFTV

SEQ ID NO: 268 - protein, 147 aa, synthetic construct
MLQEKIDHLERLLAENNEIISNIRDSVINLSESVEDGPKSSQSNFSQGAGSHLLPSQLSLSVDTADCLFASQSGSHNSDVQMLDVYSLISFDNPDGGVWKQGFDITYESNEWDTEPLQVFVVPHSHNDPGPALNVARKNRPFGAFAS

SEQ ID NO: 269 - protein, 52 aa, synthetic construct; identical to SEQ ID NO: 267

SEQ ID NO: 270 - protein, 30 aa, synthetic construct
MVEFGSKDLTLLMNLMNGTLNPFKLVEDFQ

SEQ ID NO: 271 - protein, 47 aa, synthetic construct
MVLKLKEDSRRKFIWSEISYLSKWWDIIDIQKKDAVKRASSQCCKKK

SEQ ID NO: 272 - protein, 120 aa, synthetic construct
MLQEKIDHLERLLAENNEIISNIRDSVINLSESVEDGPKSSQSNFSQGAGSHLLPSQLSLSVDTADCLFASQSGSHNSDVQMLDVYSLISFDNPDGGVWKQGFDITYESNEWDTEPLQVG

SEQ ID NO: 273 - protein, 20 aa, synthetic construct
MRWIKQMKLRETRANRCSLF

SEQ ID NO: 274 - protein, 29 aa, synthetic construct
MPIEMIITGVAILHPDPFTNEWTESWNLI

SEQ ID NO: 275 - protein, 31 aa, synthetic construct
RYSLELYQIFLMRWIKQMKLRETRANRCSLF

SEQ ID NO: 276 - protein, 133 aa, synthetic construct
MFPVLSGDFFTYADRDDHYWSGYFTSRPFYKRMDRIMESHLRLFHSLMVLEKIIGNSAFLLILKDKLTYDSYSPDTFLEMDLKQKSQDSLPQKNIIRLSAEPRYSLELYQIFLMRWIKQMKLRETRANRCSLF

SEQ ID NO: 277 - protein, 5 aa, synthetic construct
VPCGL

SEQ ID NO: 278 - protein, 17 aa, synthetic construct
MLLIESDYTTYRVPCGL

SEQ ID NO: 279 - protein, 13 aa, synthetic construct
MSLMFTQHRPLSE

SEQ ID NO: 280 - protein, 44 aa, synthetic construct
MFRSVRHMIYDLIEWRSQILSGTLPQDELKELKKKVTAKIDYGN

SEQ ID NO: 281 - protein, 47 aa, synthetic construct
MFRSVRHMIYDLIEWRSQILSGTLPQDELKELKKKVTAKIDYGNSFL

SEQ ID NO: 282 - protein, 148 aa, synthetic construct
METAFYNYDARGADELSLQIGDTVHILETYEGWYRGYTLRKKSKKGIFPASYIHLKEAIVEGKGQHETVIPGDLPLIQEVTTTLREWSTIWRQLYVQDNREMFRSVRHMIYDLIEWRSQILSGTLPQDELKELKKKVTAKIDYGNSFL

SEQ ID NO: 283 - protein, 13 aa, synthetic construct
MAITNSFWNSASG

SEQ ID NO: 284 - protein, 19 aa, synthetic construct
MMPEERMNFLYRSETLCTS

SEQ ID NO: 285 - protein, 29 aa, synthetic construct
MKGGTEVTRYEKSLRRVYFLLHIFILKKR

SEQ ID NO: 286 - protein, 43 aa, synthetic construct
MKQSSRVTSPSSRKSPRHSESGPPSGGSSTCKITGRCFEVCGT

SEQ ID NO: 287 - protein, 22 aa, synthetic construct
MTLLNGDHKFFLELCLRMNSKN

SEQ ID NO: 288 - protein, 109 aa, synthetic construct
MELRDNNTRKLTSGLRRPFGVAAFYNYDARGADELSLQIGDTVHILETYEGWYRGYTLRKKSKKDLGSKDLKREKISFVCQIVRVGRMELRDNNTRKLTSGLRRPFGVA

SEQ ID NO: 289 - protein, 18 aa, synthetic construct
MMPEERMNFLYRSETLCT

SEQ ID NO: 290 - protein, 10 aa, synthetic construct
FGVAAFYNYD

SEQ ID NO: 291 - protein, 22 aa, synthetic construct
MKGGTEVTRYEKSLRRTSEAKT

SEQ ID NO: 292 - protein, 285 aa, synthetic construct
MTFTKTSQKFGQWADSRANTVYGLGFSSEHHLSKFAEKFQEFKEAARLAKEKSQEKMELTSTPSQESAGGDLQSPLTPESINGTDDERTPDVTQNSEPRAEPTQNALPFSHREQPIFSTRAHVFQIDPNTKKNWVPTSKHAVTVSYFYDSTRNVYRIISLDGSKAIINSTITPNMTFTKTSQKFGQWADSRANTVYGLGFSSEHHLSKFAEKFQEFKEAARLAKEKSQEKMELTSTPSQESAGGDLQSPLTPESINGTDDERTPDVTQNSEPRAEPTQNALPFSH

SEQ ID NO: 293 - protein, 12 aa, synthetic construct
LPFSHREQPIFS

SEQ ID NO: 294 - protein, 52 aa, synthetic construct
MHCHFHIGNNLSSALELMSSKLTQTQRRTGYPPASMQLLCLISMTAQEMCIG

SEQ ID NO: 295 - protein, 35 aa, synthetic construct
MSSKLTQTQRRTGYPPASMQLLCLISMTAQEMCIG

SEQ ID NO: 296 - protein, 26 aa, synthetic construct
MDWDSPLSIIFRNLQKSFRNLKKLLD

SEQ ID NO: 297 - protein, 31 aa, synthetic construct
MTIKLKATGDHIYGEQLKDDRGGEQTHRTEQ

SEQ ID NO: 298 - protein, 62 aa, synthetic construct
MESNLKTIEEENKLIEQNNESLLKELAGLSQALISSLADIQLPQMLIWMSSQCTSYQKGQGF

SEQ ID NO: 299 - protein, 18 aa, synthetic construct
MLIWMSSQCTSYQKGQGF

SEQ ID NO: 300 - protein, 90 aa, synthetic construct
MYKGVIQAIQKSDEGHPFRAYLESEVAISEELVQKYSNSALGHVNCTIKELRRLFLVDDLVDSLKLLTSCTGETLRRLEWCLVPAYSCCF

SEQ ID NO: 301 - protein, 24 aa, synthetic construct
LLTSCTGETLRRLEWCLVPAYSCC

SEQ ID NO: 302 - protein, 31 aa, synthetic construct
MKATHSGHIWNLKLLYLRSWFRSTVILLLVM

SEQ ID NO: 303 - DNA, 360 bp, Homo sapiens
  source 1..360: mol_type = other DNA
ggccagctctcaatgttgcaagaaaaaatagaccatttggagcgtttgctagctgagaataatgagatcatctcaaatattagagactcagtcatcaatttgagtgagtctgtggaggatggtccgaaaagttcacaaagcaatttcagccaaggtgctggctcacatcttctgccctcacaattatccctctcagttgacactgcagactgtctgtttgcttcacaaagtggaagtcacaattcagatgtgcagatggtggagtttggaagcaaggatttgacattacttatgaatctaatgaatgggacactgaaccccttcaagtctttgtggtgcctcattcccataacgacccag

SEQ ID NO: 304 - DNA, 400 bp, Homo sapiens
  source 1..400: mol_type = other DNA
ggccagctctcaatgttgcaagaaaaaatagaccatttggagcgtttgctagctgagaataatgagatcatctcaaatattagagactcagtcatcaatttgagtgagtctgtggaggatggtccgaaaagttcacaaagcaatttcagccaaggtgctggctcacatcttctgccctcacaattatccctctcagttgacactgcagactgtctgtttgcttcacaaagtggaagtcacaattcagatgtgcagatgttggatgtttacagtctaatttcttttgacaatccagatggtggagtttggaagcaaggatttgacattacttatgaatctaatgaatgggacactgaaccccttcaagtctttgtggtgcctcattcccataacgacccag

SEQ ID NO: 305 - DNA, 538 bp, Homo sapiens
  source 1..538: mol_type = other DNA
ggccagctctcaatgttgcaagaaaaaatagaccatttggagcgtttgctagctgagaataatgagatcatctcaaatattagagactcagtcatcaatttgagtgagtctgtggaggatggtccgaaaagttcacaaagcaatttcagccaaggtgctggctcacatcttctgccctcacaattatccctctcagttgacactgcagactgtctgtttgcttcacaaagtggaagtcacaattcagatgtgcagatgttggatgtttacagtctaatttcttttgacaatccagatggtggagtttggaagcaaggatttgacattacttatgaatctaatgaatgggacactgaaccccttcaagttggttgaagactttcaatgactactttagagacaagactcagtatatttttaataacatggtcctaaagctgaaagaagactcacggaggaagtttatttggtctgagatctcttacctttcaaagtggtgggatattatagatattcagaagaaggatgctgttaaaag

SEQ ID NO: 306 - DNA, 386 bp, Homo sapiens
  source 1..386: mol_type = other DNA
atacagtttggaactttatcagatttttttgatgcgctggataaagcagatgaaactcagagagacaagggccaatcgatgttccctgttttaagtggagattttttcacttatgccgatcgagatgatcattactggagtggctattttacatccagacccttttacaaacgaatggacagaatcatggaatctcatttaagactttttcattcgttaatggttttggagaagataattggaaattctgcatttcttcttattttgaaggacaaactcacatacgactcttactctcctgataccttcctggagatggatttgaaacaaaaatcacaagattctctgccacaaaaaaatataataaggctgagtgcggagccaag

SEQ ID NO: 307 - DNA, 281 bp, Homo sapiens
  source 1..281: mol_type = other DNA
gtaccttgtggtctataatcctttagaacaagaccgaatctcgttggtctcagtctatgtgagttccccgacagtgcaagtgttctctgcttcaggaaaacctgtggaagttcaagtcagcgcagtttgggatacagcaaatactatttcagaaacagcctatgagccttatgtttacacaacaccgccctttgtcagagtgacacatggaaggatttattcggaagtgacttgcttttttgaccatgttactcatagagtccgactataccacatacagg

SEQ ID NO: 308 - DNA, 427 bp, Homo sapiens
  source 1..427: mol_type = other DNA
ctttttataactatgatgccagaggagcggatgaactttctttacagatcggagacactgtgcacatcttagaaacatatgaagggtggtaccgaggttacacgttacgaaaaaagtctaagaagggtatatttcctgcttcatatattcatcttaaagaagcgatagttgaaggaaaagggcaacatgaaacagtcatcccgggtgacctccccctcatccaggaagtcaccacgacactccgagagtggtccaccatctggaggcagctctacgtgcaagataacagggagatgtttcgaagtgtgcggcacatgatctatgaccttattgaatggcgatcacaaattctttctggaactctgcctcaggatgaactcaaagaactgaagaagaaggtcacagccaaaattgattatggaaacag

SEQ ID NO: 309 - DNA, 261 bp, Homo sapiens
  source 1..261: mol_type = other DNA
ctttttataactatgatgccagaggagcggatgaactttctttacagatcggagacactgtgcacatcttagaaacatatgaagggtggtaccgaggttacacgttacgaaaaaagtctaagaaggacctcggaagcaaagacctgaaaagggagaaaatcagttttgtctgtcagattgttcgcgtgggtcgcatggagctgagggacaacaacaccaggaaactgacctcggggttgcggcgaccttttggagtggctg

SEQ ID NO: 310 - DNA, 522 bp, Homo sapiens
  source 1..522: mol_type = other DNA
ggaacaacctatcttcagcactcgagctcatgtcttccaaattgacccaaacacaaagaagaactgggtacccaccagcaagcatgcagttactgtgtcttatttctatgacagcacaagaaatgtgtataggataatcagtttagatggctcaaaggcaataataaatagtaccatcaccccaaacatgacatttactaaaacatctcagaagtttggccagtgggctgatagccgggcaaacaccgtttatggattgggattctcctctgagcatcatctttcgaaatttgcagaaaagtttcaggaatttaaagaagctgctcgactagcaaaggaaaaatcacaagagaagatggaacttaccagtacaccttcacaggaatccgcaggcggggatcttcagtctcctttaacaccggaaagtatcaacgggacagatgatgaaagaacacctgatgtgacacagaactcagagccaagggctgaaccaactcagaatgcattgccattttcacatag

SEQ ID NO: 311 - DNA, 233 bp, Homo sapiens
  source 1..233: mol_type = other DNA
cttatctggatgtcctctcaatgcacaagttatcaaaaagggcaaggtttctgaagaactcatgaccatcaagctcaaagcaactggggatcacatctatggagagcaacttaaagacgatagaggaggagaacaaactcatagaacagaacaatgaaagtctgctgaaagagctggcaggtctaagccaagctctcatttcaagccttgctgacatccagcttccacagatg

SEQ ID NO: 312 - DNA, 347 bp, Homo sapiens
  source 1..347: mol_type = other DNA
ttgttgacctcctgtactggagagacattaagaagactggagtggtgtttggtgccagcctattcctgctgctttcattgacagtattcagcattgtgagcgtaacagcctacattgccttggccctgctctctgtgaccatcagctttaggatatacaagggtgtgatccaagctatccagaaatcagatgaaggccacccattcagggcatatctggaatctgaagttgctatatctgaggagttggttcagaagtacagtaattctgctcttggtcatgtgaactgcacgataaaggaactcaggcgcctcttcttagttgatgatttagttgattctctgaag

(19) United States
(12) Patent Application Publication
(10) Pub. No.: US 2024/0401055 A1
(43) Pub. Date: Dec. 5, 2024

(54) INHIBITION OF NEUROFIBRILLARY TANGLES USING OLIGONUCLEOTIDES AGAINST CIRCULAR RNAS FROM THE MICROTUBULE ASSOCIATED PROTEIN TAU (MAPT) LOCUS

(71) Applicant: UNIVERSITY OF KENTUCKY RESEARCH FOUNDATION, Lexington, KY (US)

(72) Inventors: Stefan Stamm, Lexington, KY (US); Justin Ralph Welden, Lexington, KY (US)

(73) Assignee: UNIVERSITY OF KENTUCKY RESEARCH FOUNDATION, Lexington, KY (US)

(21) Appl. No.: 18/291,367

(22) PCT Filed: Jul. 22, 2022
(86) PCT No.: PCT/US22/74065

§ 371 Date: Jan. 23, 2024

Related U.S. Application Data
(60) Provisional application No. 63/224,959, filed on Jul. 23, 2021.

(51) Int. Cl. C12N 15/113; A61P 25/16

(52) U.S. Cl. CPC C12N 15/1137; A61P 25/16; C12N 2310/14

(57) ABSTRACT

This disclosure relates to the development of nucleotide compositions directed to spliced gene variants that generate circular RNAs that are indicative of Alzheimer's disease. In some aspects, the nucleotides are siRNA compositions. In some aspects, the circular RNA is from a backspliced variant produced from the MAPT gene. The circular RNAs contain multiple microtubule binding domains and feature no frame shift and no stop codon when translated, allowing for consistent production. The siRNA compositions disclosed herein demonstrate good efficacy at silencing expression of the circRNAs. Circular RNAs, including tau circular RNAs exhibit a dramatic increase in translation after undergoing A>I editing, i.e. deamination of adenosines to inosines, which causes the formation and accumulation of pathological proteins. This accumulation can be prevented by siRNAs. In addition, five circular RNAs have been identified that correlate in expression levels with Alzheimer's disease severity. These circRNAs express proteins after adenosine to inosine editing. Part of these circproteins are specific for circular RNAs and could be molecular markers for Alzheimer's disease, accessible from cerebrospinal fluid.

### RELATED APPLICATIONS

This application is a U.S. national phase of International Application No. PCT/US22/74065, filed Jul. 22, 2022, which claims priority to U.S. Provisional Application Ser. No. 63/224,959 filed Jul. 23, 2021 which is incorporated herein by reference in its entirety.

### GOVERNMENT SUPPORT

This invention was made with support from grant CFDA 93.866 from the National Institute of Health AG067438 and from grant CFDA 12.420 from the Department of Defense W81XWH-19-1-0502. The government may have rights to the invention.

### SEQUENCE LISTING

The present application includes a sequence listing, which has been submitted in ASCII format, hereby incorporated by reference, entitled UKY2539SEQ.XML, having a size of 313 kB with a creation date of Apr. 24, 2024.

### TECHNICAL FIELD

This disclosure relates inhibiting the formation of neurofibrillary tangles and related tauopathies through targeting circular RNAs generated by backsplicing between exons from the microtubule associated protein tau (MAPT) locus and by targeting translation thereof and to increase the translation of MAPT and other circular RNAs through adenosine to inosine (A>I) RNA editing. This disclosure also relates to peptides expressed by circular RNAs and antibodies directed to the same for the detection and/or monitoring of disease onset and progression.

### BACKGROUND

The formation of neurofibrillary tangles (NFTs) is a hallmark of Alzheimer's disease and related tauopathies, such as frontotemporal lobar degenerations (FTLDs) and Frontotemporal dementia with Parkinsonism linked to Chromosome 17 (FTDP-17), now called frontotemporal lobar degeneration (FTPD-Tau). NFTs are composed of aggregates of microtubule associated protein tau (MAPT), which is usually hyperphosphorylated.

It has recently been identified that the MAPT gene will generate circular RNA molecules due to an identified backsplicing between exons 12 and 10 (12->10) and exons 12 and 7 (12->7) (see, Welden J R, van Doorn J, Nelson P T, Stamm S; Biochim Biophys Acta. 2018;1864 (9 Pt B): 2753-60). Both backsplicing occurrences generate circular RNA (circRNA) molecules with 12->7 having a sequence length of 681 nucleotides (nt) encoding 227 amino acids and 12->10 having a sequence length of 288 nt, encoding 96 amino acids. The 12->7 circRNA features a natural start codon. The 12->10 circRNA has no native start codon, but two identified human mutations, V337M and K317M, will introduce in frame start codons. Adenosine to inosine RNA editing of the 12->10 circRNA also causes translation, by generating AUI start codons from AUA codons. (I: Inosine). An adenosine in the AUA codon #99 is converted to inosine, generating and AUI start codon.

Both the 12->10 and the 12->7 circular RNAs do not contain stop codons and both are divisible perfectly by three. As RNA is translated in codons of three, this means when protein translation of the RNA occurs, the translation machinery can continue ad infinitum to result in formed multimers from the continuing in a ‘rolling circle’ mechanism. Significantly, the translation of the 12->10 tau circRNA generates a peptide with three identified tau microtubule binding sites (3R tau) and the translation of the 12->7 tau circRNA generates a peptide with four identified tau microtubule binding sites (4R tau). Three consecutive inosines, generated through RNA editing and new entering ribosomes at the start codon inhibit and eventually terminate the rolling circle translation.

### SUMMARY

The present disclosure concerns nucleotides and proteins/peptides related to circular RNA (circRNA) products formed from backsplicing and associated with neurological conditions and pathologies. In some aspects, the present disclosure concerns nucleotides, proteins/peptides, and antibodies to detect and or silence circRNAs, including circMAPT, circMAN2A1, circDOCK1, circHOMER1, circST18, and/or circRTN4.

In some aspects, the present disclosure concerns isolated double stranded (ds) silencing ribonucleic acid (siRNA) to silence expression of a backsplice circRNA from exon 12 of the MAPT gene comprising a fragment of exon 12 and a fragment of the backspliced exon. In some aspects, the isolated ds siRNA may include between 17 and 22 contiguous nucleic acids from SEQ ID NO: 1 and/or SEQ ID NO: 39. In some aspects, the contiguous nucleic acids may include at least one nucleic acid from exon 12 and one nucleic acid from either exon 10 or exon 7 of the MAPT gene. In some aspects, the nucleic acid sequence may be selected from the group consisting of SEQ ID NOs: 2-19 or selected from the group consisting of SEQ ID NOs: 40-57.

In some aspects, the nucleic acid sequences herein may include one or more modified nucleic acids.

In some aspects, the nucleic acids described herein may be double stranded or single stranded. In some aspects, the double strands are two separate annealed strands. In other aspects, the double strands are a self-annealed single strand.

In some aspects, the present disclosure concerns methods of treating or alleviating formation of neurofibrillary tangles through administering an isolated nucleotide in the form of a ds siRNA as disclosed herein. In some aspects, the present disclosure concerns methods to treat frontotemporal degeneration with Parkinsonism linked to chromosome 17 (FTDP17) (or FTLD-tau) through administering an isolated ds siRNA as disclosed herein. In some aspects, a nucleotide, such as ds siRNA, is administered by intrathecal injection.

In some aspects, the present disclosure concerns methods for increasing translation of a circular RNA molecule by administering to the circular RNA an adenosine deaminase acting on RNA (ADAR) activity wherein the ADAR edits at least one adenosine in the circular RNA molecule to an inosine. In some aspects, the circular RNA molecule is a tau circular RNA as described herein.

In some aspects, the present disclosure concerns methods for inhibiting translation of a circular RNA molecule in a cell by inhibiting an ADAR enzyme within the cell from editing the circular RNA molecule. In some aspects, the ADAR enzyme is inhibited by phosphorylation of at least one serine or threonine therein. In some aspects, the ADAR enzyme is phosphorylated by AKT.

In some aspects, the present disclosure concerns an isolated nucleic acid that corresponds to a circular RNA (circRNA) sequence that includes a nucleotide sequence of at least twenty contiguous nucleotides, wherein at least a terminal of the contiguous nucleotide sequence corresponds to a first exon within a gene and a second terminus of the contiguous nucleotide sequence corresponds to a second exon within the gene, the first and second exons being different. In some aspects, the contiguous nucleotide sequence is derived from a splice junction site or site of backsplicing selected from the group consisting of SEQ ID NO: 80, SEQ ID NO: 100, SEQ ID NO: 120, SEQ ID NO: 140, SEQ ID NO: 160, SEQ ID NO: 180, SEQ ID NO: 200, SEQ ID NO: 220, and SEQ ID NO: 240. In some aspects, the contiguous nucleotide sequence includes a sequence selected from SEQ ID Nos: 60-259. In some aspects, the isolated nucleotide may further include a complementary strand, such as a separate annealed strand and/or a sequence connected to the contiguous nucleotide sequence to form a self-annealed single strand. In some aspects, one or more nucleic acids therein are modified. In some aspects, the present disclosure concerns methods for treating or alleviating neurodegeneration in a subject comprising administering the isolated nucleic acids to the subject.

In some aspects, the present disclosure concerns an isolated peptide that includes an amino acid sequence selected from the group consisting of SEQ ID NOS: 260-302. In some aspects, the present disclosure includes nucleotides encoding the isolated peptides.

In some aspects, the present disclosure concerns antibodies with complementary determination regions (CDRs) that bind the isolated peptides described herein. In some aspects, the antibody is a monoclonal antibody. In some aspects, the antibody is a polyclonal antibody.

In some aspects, the present disclosure concerns methods of determining the onset and/or progression of Alzheimer's disease in a subject, through obtaining a sample from the subject and administering to the sample the antibodies as described herein. In some aspects, the methods may further include initiating treatment upon determining when the antibody binds an antigen in the sample. In some aspects, the method may further include determining a Braak score based on the concentration of antigen in the sample determined by the antibody binding. In some aspects, the methods may further include determining the concentration of antigen in the sample and obtaining a Braak score therefrom.

In some aspects, the present disclosure concerns methods for measuring a Braak score in a subject through obtaining a sample from the subject; administering to the sample the antibodies as described herein; and, determining the concentration of antibody binding within the sample, wherein higher antibody binding increases the Braak score.

### BRIEF DESCRIPTION OF THE DRAWINGS

FIG. 1 depicts an overview of the processes provided by the instant disclosure. FIG. 1A shows a predicted partial structure of the MAPT-pre mRNA. Human-specific Alu elements (triangles) fold the pre-mRNA so that backsplicing can occur (in this case the shown mechanism is the backsplicing between exon 12->10). FIG. 1B shows a schematic of the backsplicing occurring, resulting in the two 12->7 and 12->10 circular RNAs. As depicted, there is an in frame start codon (AUG/Met) in exon 9 of the 12->7 RNA. FIG. 1C shows the circular RNAs formed from the tau locus. The 12->7 RNA contains one open reading frame of 681 nucleotides and 227 amino acids. The 12->10 circular RNA has an open reading frame without a start codon that is 288 nucleotides in length, i.e. encodes 96 amino acids. Two FTDP-17 mutations, V337M and K317M introduce start codons (M: methionine). In addition, the AUA codon (Ile) in exon 9 is converted by ADAR2 to AUI, which serves as a start codon. FIG. 1D shows how both circular RNAs are translated into the depicted proteins.

FIG. 2 depicts an overview of locations utilized for siRNA knockdown expression of the circular RNA proteins. As depicted for the 12->10 circRNA, siRNA is directed against the 12->10 backsplicing junction. For 12->7, the siRNA targets the exons 12 and 7 junction. In both cases, the siRNA molecules target the bridge formed from the backsplicing events.

FIG. 3A depicts the silencing effect of the 12->10 siRNA molecules in cells expressing the protein produced by the circRNA. Cells were transfected with siRNA and harvested 48 hours later. 200 μg of lysate was immunoprecipitated with anti-Flag antibody M2, washed, loaded on each lane of a polyacrylamide gel and transferred to nitrocellulose for western blotting with antibodies directed to tau. Lanes 1-4 shows the effect of increasing concentrations of transfected siRNA, with lane 5 providing a control to green fluorescent protein and lane 6 showing a negative control. Stars represent the size of the proteins expected to be observed. Transfection with GFP or treatment with only lipofectamine serves as negative controls.

FIG. 3B depicts the effect of two different 12->7 siRNA variants on expression of the backsplice in an arrangement similar to FIG. 3A. Lanes 1-3 shows one set of 12->7 siRNA oligos and lanes 5-7 show the other. Lanes 4 and 8 present the negative control, with the stars further marking the expecting protein sizes. Transfection with GFP or treatment with only lipofectamine serves as negative controls.

FIG. 4 depicts that RNA editing caused by overexpression of the human proteins ADAR1 and ADAR2 strongly promotes translation. The protein resulting from the 12->7 backsplice is likely present in most brain parts. The protein made from 12->10 is limited to expression only when the identified mutations are present or when A->IRNA editing occurs. FIG. 4 shows that cotransfection with ADAR1 and ADAR2 dramatically increases translation of the 12->7 backsplice RNA and also cause translation of the 12->10 wild type RNA, in the absence of the start-codon introducing mutations. ADAR1 and ADAR2 catalyze the conversion of Adenosines to Inosines. Inosines are read as guanosines. ADAR3 encodes a related human enzyme that however lacks the catalytic Adenosine to inosine activity. Note that ADAR1 and 2 also increase translation from the 12->7 circTau RNA.

FIG. 5A depicts that cotransfection with ADAR1 or ADAR2 dramatically increases expression of the 12->7 backsplice circRNA. Cells were contransfected with the 12->7 circRNA along with eGFP, ADAR1, ADAR2 or ADAR3. FIG. 5A shows an immunoblot with anti-FLAG antibody probes. FIG. 5B shows an immunoblot with anti-Tau antibody probes. FIG. 5C and 5D show similar experiments for the 12->10 circ RNA harboring the V337M mutation.

FIG. 6 shows a correlation of Braak stage in Alzheimer's disease with adenosine to inosine circRNA editing in the entorhinal cortex. Adenosine to inosine editing of circRNA in the entorhinal cortex increases with each stage, but there are no changes in linear mRNA. Furthermore, the adenosine to inosine editing of linear and circRNAs in the temporal cortex show no change.

FIG. 7 shows Tau circRNA products cause NFT accumulation in biosensor cells. The protein generated from tau with authentic Alus 12->7 WT co-transfected with ADAR1 was immunoprecipitated, purified an then incubated with an RNA mix to form the tau neurofibrillary fibrils and added to cells. Tau indicator cells were treated for four days with tau fibrils made from the proteins indicated, which were added to HEK 293T Tau RD FRET Biosensor cells. FIG. 7A shows Quantification of the NFTs formed. FIG. 7B. Representative pictures of the cells. The top line shows the background GFP fluorescence, the bottom line the NFT signal. The resulting pathology was indicated in the Biosensor cells. The positive control, K18 is a synthetic peptide that features the microtubule binding domains of the linear tau protein containing the FTDP-17 mutation P301L that promotes tau aggregation and pathology (FIG. 7B). Negative controls are protein made from UPFI circular RNAs, in the presence and absence of the kinase DYRKIA.

FIG. 8 shows that the RNA editing and translation and/or expression of the circRNAs can be inhibited by AKT activity. As the protein generated by ADAR editing of cicrRNAs can form tau pathology, inhibiting activity on ADARs by AKT is a further pathway to prevent NFTs and paired helical filaments. A: The 12->7 circ tau RNA was cotransfected with GFP, ADAR1 and ADAR1+AKT1. The detection of the protein was with anti Flag (A) and anti tau (B). C: A similar experiment was performed for the 12->10 circ tau RNA with out start codon. C: detection with anti flag; D; detection with anti tau. The letters a-f refer to the proteins formed by different rounds of rolling circle translation.

FIG. 9 shows that circRNAs in general are translated by adenosine to inosine editing by ADAR enzymes. Circular RNAs for (A) UPF1, (B) ADAR1 and (C) SF3B1, all with FLAG inserts, were cotransfected with either GFP or ADAR1 and immunoprecipiated with anti-FLAG and then immunoblotted for the presence of FLAG. Cotransfection with ADAR1 caused the translation of each circRNA. The exon-intron organization of the circular RNAs is indicated and the circle forms are indicated below.

FIG. 10 shows the correlation in circRNA levels with Braak stages along with depicted splice variants of A) MAN2A1; B) DOCK1; C) Homer1; D) ST18; and, E) RTN4. circRNA expression is depicted as fragments per kilo million reads (FPKM), where fragment refers to backsplice junction read. Braak stages were determined post mortem. The tissue was entorhinal cortex.

### DETAILED DESCRIPTION

### Backspliced circRNA Silencing

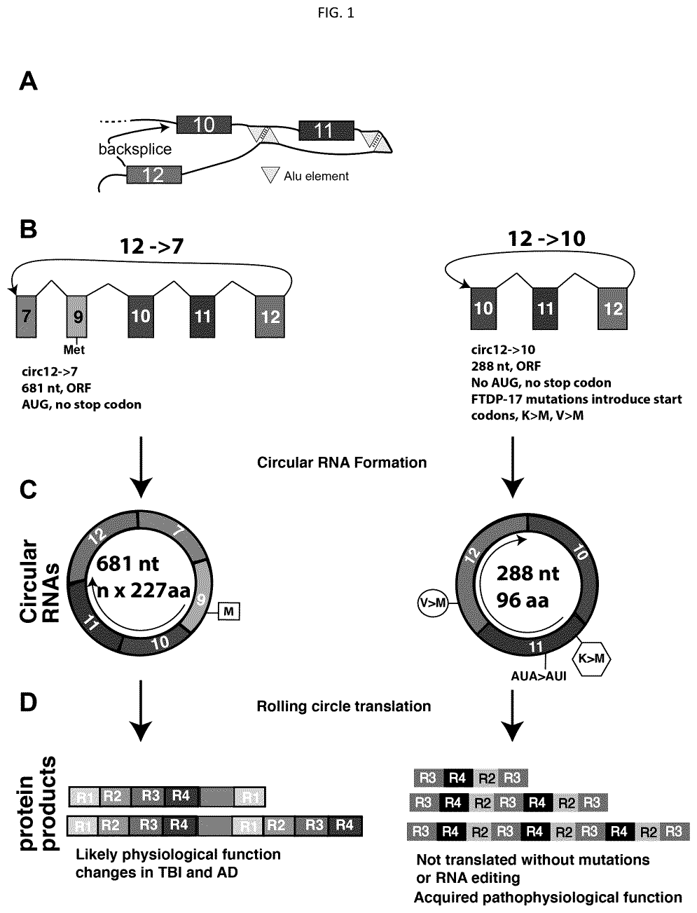

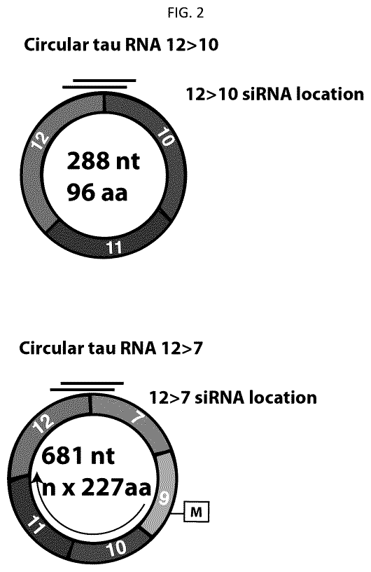

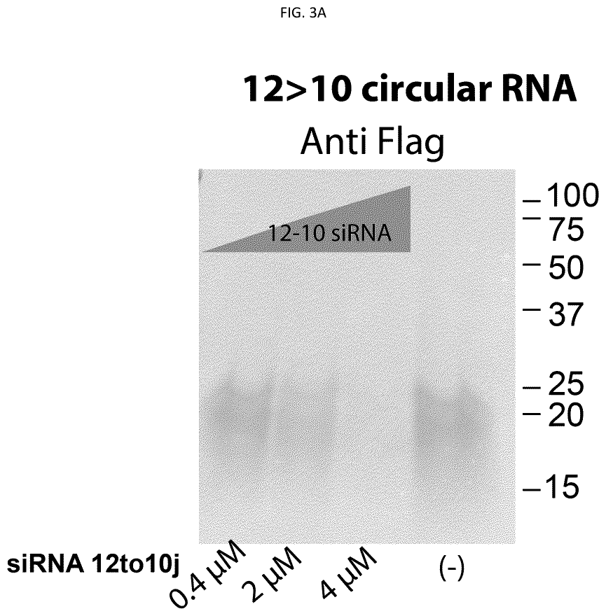

The present disclosure concerns the identification of RNA sequences that can silence and/or reduce the presence of the two circular RNA (circRNA) molecules in a cell caused by the backsplicing between exons 12 and 10 (12->10) and exons 12 and 7 (12->7) in the microtubule-associated protein tau (MAPT) gene. FIG. 1 sets forth a depiction of the backsplicing events (see, e.g. FIGS. 1A and 1B), the resulting circRNA structures (FIG. 1C) and expected protein products (FIG. 1C). As further set forth in FIG. 1, the 12->10 circRNA does not contain a natural methionine, but two possible mutations at V337 and K317 to methionines will result in a transcribed protein. The 12->7 circRNA does include a methionine in exon 9 that will allow for translation naturally. RNA adenosine to inosine (A->I) editing causes translation of the 12->10 circRNA and increases translation of the 12->7 circRNA.

In some aspects, the present disclosure concerns nucleotide sequences and double-stranded nucleotide sequences thereof that are of from between about 19 and 27 nucleotides in length, including 20, 21, 22, 23, 24, 25, and 26 nucleotides in length. In some aspects, the nucleotide sequences bridge over a backsplice site or the point where an exon backsplices to an earlier exon. For example, with 12->10 circRNA where exon 12 backsplices to exon 10, at least one nucleotide will be from exon 12 and at least one nucleotide will be from exon 10 of the MAPT gene. Similarly, with 12->7 circRNA where exon 12 backsplices to exon 7, at least one nucleotide will be from exon 12 and at least one nucleotide will be from exon 7 of the MAPT gene.

In some aspects, the present disclosure concerns nucleotide sequences that bridge between a backsplice junction between exon 12 and exon 10, wherein exon 10 is presented in bold in the sequence set forth in SEQ ID NO: 1:

ACCCACGTCCCTGGCGGAGGAAATAAAAAGGTGCAGATAATTAATAAGAAGCTG GATCTTAGCAACGTCCAGTCCAAGTGTG (SEQ ID NO: 1). It will also be appreciated that nucleotides derived from such when in a deoxyribonucleic acid format will include thymine, whereas as nucleotide sequences in a ribonucleic acid format will include uracil residues in the place of thymidine residues. For the purposes of SEQ ID NO: 1, T can be interpreted as either thymine or uracil.

In some aspects, the nucleotide sequences of the present disclosure cover the identified bridge between the two exons over a “segment of interest.” Such may refer to a span or a sequence of consecutive nucleic acids within SEQ ID NO: 1 that are to be provided to a cell or transcribed therein to effectuate the desired silencing. In some aspects, the segment of interest may include between about 17 and about 27 contiguous nucleotides that span the exon junction (show as a space) from

| SEQ ID NO: 76: | |
|---|---|
| 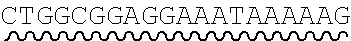 | GTGCAGATAATTAATAAGA |

(T can be interpreted as either thymine
or uracil).

It is generally understood in the art that for successful silencing in mammalian cells, the siRNA should be of sufficient length to be specific to the gene being targeted, yet also restricted in length to avoid unwanted effects, such as an interferon response. Accordingly, the present disclosure in some aspects, includes nucleotide sequences derived from SEQ ID NO: 1 of between 19 and 27 nucleotides in length, wherein the sequence includes the 3′ most unboldened guanosine and the 5′ most guanosine (or the guanosines either side of the space in SEQ ID NO: 76), such that the sequence includes the bridge between exon 12 and exon 10. By way of example, and not to be viewed as a limitation in length, suitable 5′-3′ ribonucleic acid sequences can include (with exon 10 in bold and a space provided for the bridge or point of backsplicing):

(SEQ ID NO: 2)
G GUGCAGAUAAUUAAUAAGnn
(SEQ ID NO: 3)
AG GUGCAGAUAAUUAAUAAnn
(SEQ ID NO: 4)
AAG GUGCAGAUAAUUAAUAnn
(SEQ ID NO: 5)
AAAG GUGCAGAUAAUUAAUnn
(SEQ ID NO: 6)
AAAAG GUGCAGAUAAUUAAnn
(SEQ ID NO: 7)
AAAAAG GUGCAGAUAAUUAnn
(SEQ ID NO: 8)
UAAAAAG GUGCAGAUAAUUnn
(SEQ ID NO: 9)
AUAAAAAG GUGCAGAUAAUnn
(SEQ ID NO: 10)
AAUAAAAAG GUGCAGAUAAnn
(SEQ ID NO: 11)
AAAUAAAAAG GUGCAGAUAnn
(SEQ ID NO: 12)
GAAAUAAAAAG GUGCAGAUnn
(SEQ ID NO: 13)
GGAAAUAAAAAG GUGCAGAnn
(SEQ ID NO: 14)
AGGAAAUAAAAAG GUGCAGnn
(SEQ ID NO: 15)
GAGGAAATAAAAAG GTGCAnn
(SEQ ID NO: 16)
GGAGGAAATAAAAAG GTGCnn
(SEQ ID NO: 17)
CGGAGGAAATAAAAAG GTGnn
(SEQ ID NO: 18)
GCGGAGGAAATAAAAAG GTnn
(SEQ ID NO: 19)
GGCGGAGGAAATAAAAAG Gnn

It will be appreciated that at the 3′ end of any sequence presented herein, the motif “nn” is optional as an overhang for siRNA. In some aspects, the “nn” motif may be of two thymidine residues. (See, e.g., Park et al. Nucleic Acid Ther. 24(5): 364-371 (2014)). It will also be appreciated that the nucleotide sequences can include the antisense of the above, such as (going 3′-5′ with 5′-3′ orientation in parentheses):

(SEQ ID NO: 20)
nnC CACGUCUAUUAAUUAUUC (CUUAUUAAUUAUCUGCAC Cnn)
(SEQ ID NO: 21)
nnUC CACGUCUAUUAAUUAUU UUAUUAAUUAUCUGCAC CUnn)
(SEQ ID NO: 22)
nnUUC CACGUCUAUUAAUUAU (UAUUAAUUAUCUGCAC CUUnn)
(SEQ ID NO: 23)
nnUUUC CACGUCUAUUAAUUA (AUUAAUUAUCUGCAC CUUUnn)
(SEQ ID NO: 24)
nnUUUUC CACGUCUAUUAAUU (UUAAUUAUCUGCAC CUUUUnn)
(SEQ ID NO: 25)
nnUUUUUC CACGUCUAUUAAU (UAAUUAUCUGCAC CUUUUUnn)
(SEQ ID NO: 26)
nnAUUUUUC CACGUCUAUUAA (AAUUAUCUGCAC CUUUUUAnn)
(SEQ ID NO: 27)
nnUAUUUUUC CACGUCUAUUA (AUUAUCUGCAC CUUUUUAUnn)
(SEQ ID NO: 28)
nnUUAUUUUUC CACGUCUAUU (UUAUCUGCAC CUUUUUAUUnn)
(SEQ ID NO: 29)
nnUUUAUUUUUC CACGUCUAU (UAUCUGCAC CUUUUUAUUUnn)
(SEQ ID NO: 30)
nnCUUUAUUUUUC CACGUCUA (AUCUGCAC CUUUUUAUUUCnn)
(SEQ ID NO: 31)
nnCCUUUAUUUUUC CACGUCU (UCUGCAC CUUUUUAUUUCCnn)
(SEQ ID NO: 32)
nnUCCUUUAUUUUUC CACGUC (CUGCAC CUUUUUAUUUCCUnn)
(SEQ ID NO: 33)
nnCUCCUUUAUUUUUC CACGU (UGCAC CUUUUUAUUUCCUCnn)
(SEQ ID NO: 34)
nnCCUCCUUUAUUUUUC CACG (GCAC CUUUUUAUUUCCUCCnn)
(SEQ ID NO: 35)
nnGCCUCCUUUAUUUUUC CAC (CAC CUUUUUAUUUCCUCCGnn)
(SEQ ID NO: 36)
nnCGCCUCCUUUAUUUUUC CA (AC CUUUUUAUUUCCUCCGCnn)
(SEQ ID NO: 37)
nnCCGCCUCCUUUAUUUUUC C (C CUUUUUAUUUCCUCCGCCnn).

Accordingly, in some aspects, the present disclosure concerns paired nucleotide sequences, either as two separate strands and/or as two annealed separate strands and/or as part of a continuous strand such that the strand may fold on itself and self-anneal, such as with a hairpin loop segment of non-matching bases between the two. For example, SEQ ID NO: 2 can pair with SEQ ID NO: 20; SEQ ID NO: 3 can pair with SEQ ID NO: 21; SEQ ID NO: 4 can pair with SEQ ID NO: 22; SEQ ID NO: 5 can pair with SEQ ID NO: 23; SEQ ID NO: 6 can pair with SEQ ID NO: 24; SEQ ID NO: 7 can pair with SEQ ID NO: 25; SEQ ID NO: 8 can pair with SEQ ID NO: 26; SEQ ID NO: 9 can pair with SEQ ID NO: 27; SEQ ID NO: 10 can pair with SEQ ID NO: 28; SEQ ID NO: 11 can pair with SEQ ID NO: 29; SEQ ID NO: 12 can pair with SEQ ID NO: 30; SEQ ID NO: 13 can pair with SEQ ID NO: 31; SEQ ID NO: 14 can pair with SEQ ID NO: 32; SEQ ID NO: 15 can pair with SEQ ID NO: 33; SEQ ID NO: 16 can pair with SEQ ID NO: 34; SEQ ID NO: 17 can pair with SEQ ID NO: 35; SEQ ID NO: 18 can pair with SEQ ID NO: 36; and, SEQ ID NO: 19 can pair with SEQ ID NO: 37.

It will be further understood that any of SEQ ID NOS: 2-37 may include about 1 to about 10 further consecutive nucleic acids at either or both termini from the corresponding longer sequences set forth in SEQ ID NO: 1 and/or SEQ ID NO: 76, including 2, 3, 4, 5, 6, 7, 8, and 9 additional nucleic acids.

Similarly, with the present disclosure concerns nucleotide sequences that bridge between the backsplice junction between exon 12 and exon 7, wherein exon 7 is presented in bold in the sequence set forth in SEQ ID NO: 39: ACCCACGTCCCTGGCGGAGGAAATAAAAAGGGGGCTGATGGTAAAACGAAGATC GCCACACCGCGGGGAGCAGCCCCTCCAGGCCA (T can be interpreted as either thymine or uracil). It will also be appreciated that nucleotides derived from such when in a deoxyribonucleic format will include thymidine, whereas as nucleotide sequences in a ribonucleic acid format will include uracil residues in the place of thymidine residues.

In some aspects, the nucleotide sequences of the present disclosure cover the identified bridge between the two exons over a segment of interest with 12->7. Such may refer to a span or a sequence of consecutive nucleic acids within SEQ ID NO: 39 that are to be provided to a cell or transcribed therein to effectuate the desired silencing. In some aspects, the segment of interest may include between about 17 and about 27 contiguous nucleotides that span the exon bridge (show as a space) from

SEQ ID NO: 77:

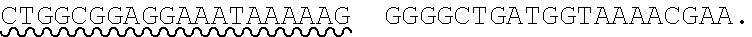

Accordingly, the present disclosure in some aspects, includes nucleotide sequences derived from SEQ ID NO: 39 of between 19 and 27 nucleotides in length, wherein the sequence includes the 3′ most non-bolded guanosine and the 5′ most guanosine (or the guanosines either side of the space in SEQ ID NO: 77), such that the sequence includes the bridge between exon 12 and exon 7. By way of example, and not to be viewed as a limitation in length, suitable 5′-3′ nucleotide sequences can include (with exon 7 in bold and a space provided for the bridge or point of backsplicing):

(SEQ ID NO: 40)

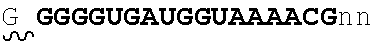

(SEQ ID NO: 41)

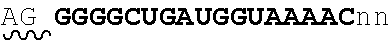

(SEQ ID NO: 42)

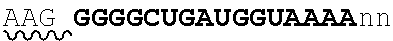

(SEQ ID NO: 43)

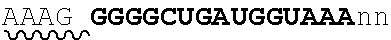

(SEQ ID NO: 44)

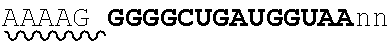

(SEQ ID NO: 45)
AAAAAG GGGGCUGAUGGUAnn
(SEQ ID NO: 46)
UAAAAAG GGGGCUGAUGGUnn
(SEQ ID NO: 47)
AUAAAAAG GGGGCUGAUGGnn
(SEQ ID NO: 48)
AAUAAAAAG GGGGCUGAUGnn
(SEQ ID NO: 49)
AAAUAAAAAG GGGGCUGAUnn
(SEQ ID NO: 50)
GAAAUAAAAAG GGGGCUGAnn
(SEQ ID NO: 51)
GGAAAUAAAAAG GGGGCUGnn
(SEQ ID NO: 52)
AGGAAAUAAAAAG GGGGCUnn
(SEQ ID NO: 53)
GAGGAAAUAAAAAG GGGGCnn
(SEQ ID NO: 54)
GGAGGAAAUAAAAAG GGGGnn
(SEQ ID NO: 55)
CGGAGGAAAUAAAAAG GGGnn
(SEQ ID NO: 56)
GCGGAGGAAAUAAAAAG GGnn
(SEQ ID NO: 57)
GGCGGAGGAAAUAAAAAG Gnn

In other aspects, the nucleotide sequences can include the antisense of the above, such as (going 3′-5′ with 5′-3′ in parentheses):

(SEQ ID NO: 58)

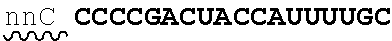

(CGUUUUACCAUCAGCCCC Cnn)
(SEQ ID NO: 59)

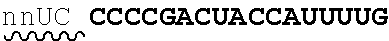

(GUUUUACCAUCAGCCCC CUnn)
(SEQ ID NO: 60)

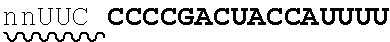

(UUUUACCAUCAGCCCC CUUnn)
(SEQ ID NO: 61)

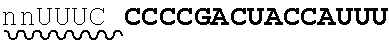

(UUUACCAUCAGCCCC CUUUnn)
(SEQ ID NO: 62)

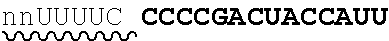

(UUACCAUCAGCCCC CUUUUnn)
(SEQ ID NO: 63)

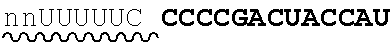

(UACCAUCAGCCCC CUUUUUnn)
(SEQ ID NO: 64)

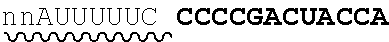

(ACCAUCAGCCCC CUUUUUAnn)
(SEQ ID NO: 65)

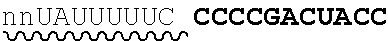

(CCAUCAGCCCC CUUUUUAUnn)
(SEQ ID NO: 66)

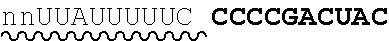

(CAUCAGCCCC CUUUUUAUUnn)
(SEQ ID NO: 67)

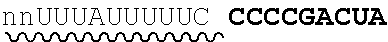

(AUCAGCCCC CUUUUUAUUUnn)
(SEQ ID NO: 68)

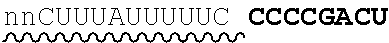

(UCAGCCCC CUUUUUAUUUCnn)
(SEQ ID NO: 69)

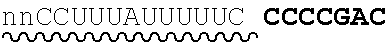

(CAGCCCC CUUUUUAUUUCCnn)
(SEQ ID NO: 70)

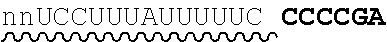

(AGCCCC CUUUUUAUUUCCUnn)
(SEQ ID NO: 71)

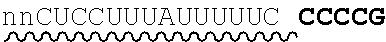

(GCCCC CUUUUUAUUUCCUCnn)
(SEQ ID NO: 72)

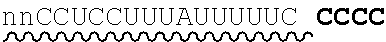

(CCCC CUUUUUAUUUCCUCCnn)
(SEQ ID NO: 73)

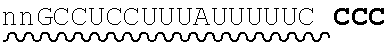

(CCC CUUUUUAUUUCCUCCGnn)
(SEQ ID NO: 74)

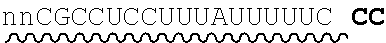

(CC CUUUUUAUUUCCUCCGCnn)
(SEQ ID NO: 75)

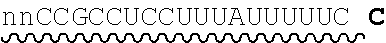

(C CUUUUUAUUUCCUCCGCCnn).

Accordingly, in some aspects, the present disclosure also concerns paired nucleotide sequences covering 12->7, either as two separate strands and/or two annealed separate strands and/or as part of a continuous strand such that the strand may fold on itself, such as with a hairpin loop segment of non-matching bases between the two. For example, SEQ ID NO: 40 can pair with SEQ ID NO: 58; SEQ ID NO: 41 can pair with SEQ ID NO: 59; SEQ ID NO: 42 can pair with SEQ ID NO: 60; SEQ ID NO: 43 can pair with SEQ ID NO: 61; SEQ ID NO: 44 can pair with SEQ ID NO: 62; SEQ ID NO: 45 can pair with SEQ ID NO: 63; SEQ ID NO: 46 can pair with SEQ ID NO: 64; SEQ ID NO: 47 can pair with SEQ ID NO: 65; SEQ ID NO: 48 can pair with SEQ ID NO: 66; SEQ ID NO: 49 can pair with SEQ ID NO: 67; SEQ ID NO: 50 can pair with SEQ ID NO: 68; SEQ ID NO: 51 can pair with SEQ ID NO: 69; SEQ ID NO: 52 can pair with SEQ ID NO: 70; SEQ ID NO: 53 can pair with SEQ ID NO: 71; SEQ ID NO: 54 can pair with SEQ ID NO: 72; SEQ ID NO: 55 can pair with SEQ ID NO: 73; SEQ ID NO: 56 can pair with SEQ ID NO: 74; and, SEQ ID NO: 57 can pair with SEQ ID NO: 75.

It will be further understood that any of SEQ ID NOS: 40-75 may include about 1 to about 10 further consecutive nucleic acids at either or both termini from the corresponding longer sequences set forth in SEQ ID NO: 39 and/or SEQ ID NO: 77, including 2, 3, 4, 5, 6, 7, 8, and 9 additional nucleic acids.

In some aspects, the paired nucleotide strands can be provided as a double stranded composition of two annealed single-strands or a single self-annealed single stand, wherein a spacer of non-matching nucleotides is provided to allow for the single strand to fold back on itself without creating strain. A spacer may be of between about 3 to about 20 unmatching nucleotides in length, including 4, 5, 6, 7, 8, 9, 10, 11, 12, 13, 14, 15, 16, 17, 18, and 19 nucleotides in length. For example, the spacer can provide a hairpin loop in a single stranded nucleotide sequence. By way of example only, a composition may include SEQ ID NO: 9 and SEQ ID NO: 27 and/or SEQ ID NO: 52 and SEQ ID NO: 70 to silence the 12->10 and/or 12->7 circRNAs, respectively. Such compositions may include SEQ ID NO: 9 and SEQ ID NO: 27 and/or SEQ ID NO: 52 and SEQ ID NO: 70 as single strands and/or as annealed separate strands and/or as a unitary single strand, optionally separated by a spacer sequence. Those skilled in the art will appreciate that in a single-stranded self-annealing sequence, SEQ ID NO: 27 and SEQ ID NO: 70 would be reversed to provide such in a unitary 5′-3′ sequence.

In some aspects, the compositions may include at least one matching pair of sequences, either as two separate strands, two annealed separate strands or a single stranded arranged to self-anneal. In other aspects, two or more matching sequences may be included, such as SEQ ID NO: 2 with SEQ ID NO: 18 and SEQ ID NO: 9 with SEQ ID NO: 27 and so on. In such combinations, each may be provided separately as single strands, as two annealed single strands, and/or as a self-annealing single strand. Accordingly, compositions may include matching separate single strands and/or two annealed single strands and/or a self-annealed single strand.

In some aspects, the nucleotide sequences of the present disclosure can include a 5′ capped RNA, such as an RNA 7-methylguanosine cap or an RNA m7G cap. In some aspects, one stand of a two-stranded annealed complex is capped. In other aspects, both strands may be capped. In instances where the nucleotide sequence is a single-stranded self-annealing strand, the 5′ end of the strand map optionally be capped.

In some aspects, the nucleic acids as set forth herein may include synthetic and/or recombinant RNA nucleic acid sequences. Recombinant RNA sequences may be produced by introducing an expression vector or a cassette into a cell that includes a promoter operably linked to a nucleic acid sequence of RNA sequences as set forth herein to allow transcription through techniques understood in the art. Recombinant RNA can also be produced by introducing a viral vector, such as an adenovirus or adeno-associated virus, to a cell with the RNA nucleic acids sequences to be transcribed operably linked to a promoter. Synthetic nucleic acid sequences may similarly be produced using techniques known in the art, such as solid phase synthesis. In some aspects, the nucleic acids of the present disclosure may be produced by other means, such as in vitro transcription that can utilize a linear DNA with the sequence to be transcribed operably linked to a promoter. In either recombinant or synthetic methods, the RNA can be expressed as two separate strands or as a single, self-annealing strand. A self-annealing strand may include a spacer between complementary strands to provide a small hairpin loop, such that the strand is able to fold onto itself and self-anneal.

In some aspects of the present disclosure, the nucleic acids as set forth herein may include one or more modified nucleic acid bases and/or a xeno nucleic acids. Such may include 5′ phosphorylation for recognition by the silencing complex, backbone modifications (phosphotriester substitutions and/or phosphorothioate/boranophosphate/phosphonoacetate linkages), sugar modifications, base modifications and/or lipid conjugations. For example, a nucleic acid base may be nucleoside modified through the incorporation of modified bases, such as pseudouridine, 1-methylpseudouridine, 5-methlycytidine, N6-methyl adenosine, 2-thio-uridine, and 5-methoxyuridine. Other modifications include 2′fluorination, 2′O-methylation, 2′O-ethylation, locked nucleic acids, C7 adenine or guanosine modifications, C5 uridine or cytosine modifications, incorporation of 2′ fluoro arabinose, incorporation of alkyl phosphonate nucleic acids, incorporation of 2′-deoxyxylnucleic acids, peptide or amino acid conjugated bases, and/or use of a phosphothionate or borano-phosphate moiety. See, e.g., Duffy et al. BMC Biol. 18, 112, (2020) and Chernikov et al. Front. Pharmacol. 10:444 doi: 10.3389/fphar.2019.00444, 2019.

In some aspects, the siRNA compositions may include a carrier or be encapsulated to allow for delivery within a cell. It is understood in the art that encapsulating nucleotide sequences within a lipid carrier may provide a means for traversing the cell membrane and delivering the nucleotides of the present disclosure to the intercellular space where they can then be recognized and trigger silencing of the circRNA genes therein. Such may include lipid nanoparticles (LNPs) or lipid-like nanoparticles (LLNs) that contain at least one the siRNA molecules or strands therein. In some aspects, the lipids may include 2,3-diolcoyloxy-N-[2 (sperminecarboxamido)ethyl]-N,N-dimethyl-1-propaniminium trifluoroacetate and 1,2,dioleoyl-sn-glycero-3-phosphoethanolamine or lipofectamine that form liposomes around the siRNA molecules. In some aspects, the siRNA may be encapsulated in an LNP or LLN of two or more lipids, such as three, four, five or more. An LNP may include an ionizable lipid (typically of three sections of an amine head, a linker and a hydrophobic tail). In some aspects, the LNP may include an ionizable lipid, a polyethylene glycol and a cholesterol. In further aspects, the LNP may include a combination of an ionizable lipid with polyethylene glycol (PEG), cholesterol and/or distearoyl phosphocholine. In some aspects, an LNP may further include one or more “helper” lipids, such as 1,2-dioleoyl-3-trimethylammoniumpropane (DOTAP) and/or dioleoylphosphatidylethanolamine (DOPE) and/or lipofectamine and/or dioleoylphosphatidylcholine (DOPC).

In some aspects, the siRNA compositions may include a further vehicle for improving cellular uptake. Such may include the presence within the compositions of one or more of an additional polymer, a polymer modified with fatty chains, a polymethacrylate with amine-bearing side chains, and a poly (beta-amino) ester (PBAE). In some aspects, the vehicle may include a dendrimer. In some aspects, the siRNA compositions may also include the presence of a cell-penetrating peptide (CPP) or a carrier protein to assist as a vector for delivery to a cell.

The present disclosure further includes methods of administering the siRNA compositions to a cell. A cell may be in vitro, ex vivo, or in vivo. As discussed herein, the siRNA compositions may feature lipids, such as cationic lipids, that encapsulate the siRNA strand(s) to assist in transporting the siRNA into the intercellular space of a cell where it can exert its silencing effect. As identified herein, the circRNAs are backspliced variants from the MAPT gene that further contain no stop codon and thus almost ceaselessly produce microtubule binding sites within neuronal networks, possibly leading to tauopathies. However, with the vehicles associated with the siRNA compositions can deliver the siRNA to the cell, it remains that the compositions need to access the extracellular space within the central nervous system. Accordingly, while typical routes of administration, such as intravenous, oral, sublingual, and other systemic routes are contemplated, it is further contemplated that for effective delivery to the central nervous system of a subject in vivo, such as an animal subject including a human, that intrathecal delivery, such as through spinal injection, may be necessary. Other routs of administration may include nasal delivery to the central nervous system (CNS) and intraventricular delivery, either through injection or through the use of an osmotic pump.

In some aspects, the present disclosure concerns methods of administering a therapeutically effective amount the siRNA compositions to a subject. A subject may include an animal, such as a mammal, including primates and humans. Such administration may be by any route, including intrathecal. Administration to a subject may additionally include the presence of a pharmaceutically acceptable carrier. Such are understood in the art and further set forth in, e.g., Remington: The Science and Practice of Pharmacy, Lippincott Williams & Wilkins, 21st Ed., 2005.

In some aspects, the present disclosure concerns methods of preventing and/or reducing neurofibrillary tangle formation and/or accumulation of such in a subject. Such methods may include delivering a 12->7 and/or a 12->10 siRNA composition as set forth herein to a subject. The siRNA can then effectively silence or reduce the translation of circRNAs and negatively impact the formation of NFTs in a subject.

In some aspects, the present disclosure concerns methods of treating and/or alleviating neurological conditions and/or symptoms associated with such by administering a therapeutically effective amount of the 12->7 and/or 12->10 siRNA compositions as set forth herein. Such may silencing and/or reduce the production of translated mictrotuble binding domains and alleviate related tauopathies and/or other conditions related with such. Accordingly, administration of the siRNA compositions to a subject may provide methods to treat and/or alleviate disease conditions associated with tau such as Alzheimer's, frontotemporal dementia with Parkinsonism-17 (FTDP17), progressive supranuclear palsy, frontoemporal lobar degeneration, Pick's disease, argyrophilic grain disease, and/or corticobasal degeneration. In further aspects, the methods of treating and/or administering the siRNA compositions may be protective and/or inhibitory for aggregation of tau, such as may be seen following an injury to the brain. In further aspects, administration of the siRNA compositions as set forth herein may be protective from tau aggregations following a traumatic brain injury.

In some aspects, the present disclosure concerns administering the siRNA compositions as set forth herein to silence the production and/or presence of the circRNAs 12->7 and/or 12->10 of the MAPT gene. In some aspects, the methods may including co-administration of the siRNAs either simultaneously, in sequence or separately with other therapeutic compounds to treat and/or alleviate symptoms associated with or caused by Alzheimer's disease or other NFT-associated conditions. Such agents may include lithium, donepezil, rivastigmine, galantamine, mematine, and combinations thereof. Similarly, the siRNA compositions may be co-administered, either simultaneously, separately or in sequence with other siRNA compositions targeting related proteins and/or pathways, such that in combination, multiple components of a dysregulated system, such as that seen with NFT progression can be targeted to prevent or alleviate any disease progression on multiple fronts within the central nervous system of a subject.

### CircRNAs From the MAN2A1, DOCK, HOMER1, ST18, and/or RTN4 Genes

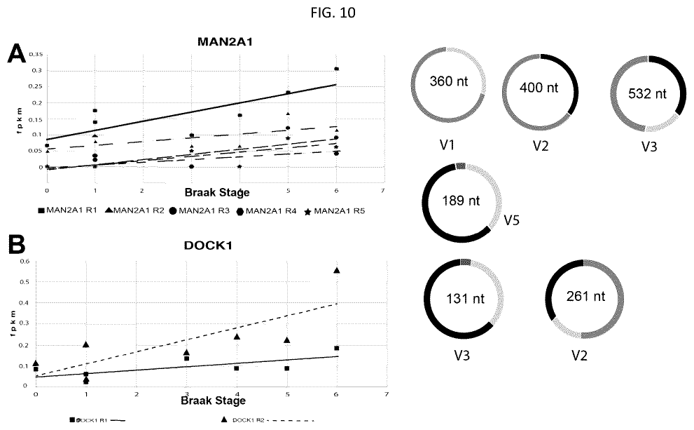

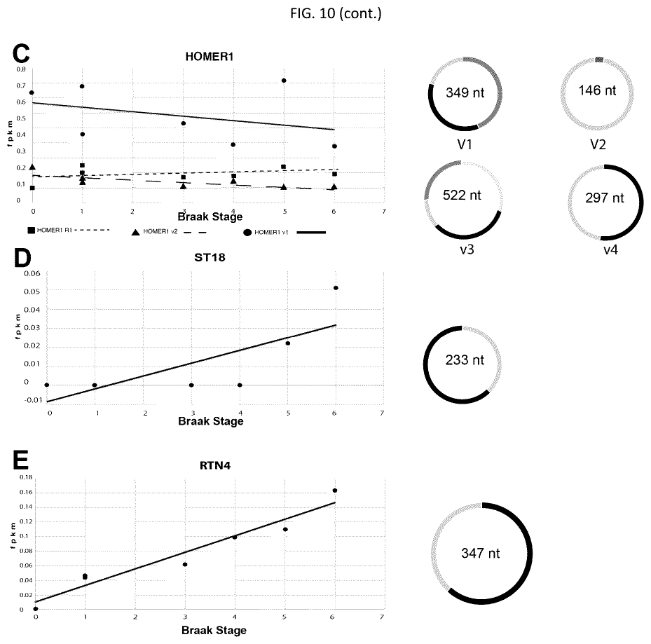

In some aspects, the present disclosure concerns nucleotides and peptides concerning circRNA for the MAN2A1, DOCK1, HOMER1, ST18, and RTN4 genes. These circular RNAs significantly change their expression during the progression of Alzheimer's disease in enthorinal cortex, the brain area where Alzheimer's disease starts (FIG. 10). The MAN2A1, DOCK1 and HOMER1 genes generate multiple circRNAs due to variable exon usage in the backsplicing events. ST18 and RTN4 generate only one circRNA.

In some aspects, the present disclosure concerns RNA sequences targeting splice junctions for the formation of additional circRNAs whose presence can be correlated to Alzheimer's disease onset and/or progression. In some aspects, the present disclosure concerns single or double stranded RNA sequence that target the splice junction of one or more of MAN2A1, DOCK, HOMER1, ST18, and/or RTN4.

In some aspects, the present disclosure concerns a splice junction in gene MAN2A1. In some aspects the splice junction include the sequence as set forth in SEQ ID NO: 80 (tcattcccataacgacccagGGCCAGCTCTCAATGTTGCA with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 81-99:

SEQ ID NO. 81
gGGCCAGCTCTCAATGTTGCnn
SEQ ID NO. 82
agGGCCAGCTCTCAATGTTGnn
SEQ ID NO. 83
cagGGCCAGCTCTCAATGTTnn
SEQ ID NO. 84
ccagGGCCAGCTCTCAATGTnn
SEQ ID NO. 85
cccagGGCCAGCTCTCAATGnn
SEQ ID NO. 86
acccagGGCCAGCTCTCAATnn
SEQ ID NO. 87
gacccagGGCCAGCTCTCAAnn
SEQ ID NO. 88
cgacccagGGCCAGCTCTCAnn
SEQ ID NO. 89
acgacccagGGCCAGCTCTCnn
SEQ ID NO. 90
aacgacccagGGCCAGCTCTnn
SEQ ID NO. 91
taacgacccagGGCCAGCTCnn
SEQ ID NO. 92
ataacgacccagGGCCAGCTnn
SEQ ID NO. 93
cataacgacccagGGCCAGCnn
SEQ ID NO. 94
ccataacgacccagGGCCAGnn
SEQ ID NO. 95
cccataacgacccagGGCCAnn
SEQ ID NO. 96
tcccataacgacccagGGCCnn
SEQ ID NO. 97
ttcccataacgacccagGGCnn
SEQ ID NO. 98
attcccataacgacccagGGnn
SEQ ID NO. 99
cattcccataacgacccagGnn

In some aspects, the junction in MAN2A1 is set forth in SEQ ID NO: 100 (aagaaggatgctgttaaaagGGCCAGCTCTCAATGTTGCA with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 101-119:

SEQ ID NO. 101
gGGCCAGCTCTCAATGTTGCnn
SEQ ID NO. 102
agGGCCAGCTCTCAATGTTGnn
SEQ ID NO. 103
aagGGCCAGCTCTCAATGTTnn
SEQ ID NO. 104
aaagGGCCAGCTCTCAATGTnn
SEQ ID NO. 105
aaaagGGCCAGCTCTCAATGnn
SEQ ID NO. 106
taaaagGGCCAGCTCTCAATnn
SEQ ID NO. 107
ttaaaagGGCCAGCTCTCAAnn
SEQ ID NO. 108
gttaaaagGGCCAGCTCTCAnn
SEQ ID NO. 109
tgttaaaagGGCCAGCTCTCnn
SEQ ID NO. 110
ctgttaaaagGGCCAGCTCTnn
SEQ ID NO. 111
gctgttaaaagGGCCAGCTCnn
SEQ ID NO. 112
tgctgttaaaagGGCCAGCTnn
SEQ ID NO. 113
atgctgttaaaagGGCCAGCnn
SEQ ID NO. 114
gatgctgttaaaagGGCCAGnn
SEQ ID NO. 115
ggatgctgttaaaagGGCCAnn
SEQ ID NO. 116
aggatgctgttaaaagGGCCnn
SEQ ID NO. 117
aaggatgctgttaaaagGGCnn
SEQ ID NO. 118
gaaggatgctgttaaaagGGnn
SEQ ID NO. 119
agaaggatgctgttaaaagGnn

In some aspects, the junction in MAN2A1 is set forth in SEQ ID NO: 120 (aggctgagtgeggagccaagATACAGTTTGGAACTTTATC with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 121-139:

SEQ ID NO. 121
gATACAGTTTGGAACTTTATnn
SEQ ID NO. 122
agATACAGTTTGGAACTTTAnn
SEQ ID NO. 123
aagATACAGTTTGGAACTTTnn
SEQ ID NO. 124
caagATACAGTTTGGAACTTnn
SEQ ID NO. 125
ccaagATACAGTTTGGAACTnn
SEQ ID NO. 126
gccaagATACAGTTTGGAACnn
SEQ ID NO. 127
agccaagATACAGTTTGGAAnn
SEQ ID NO. 128
gagccaagATACAGTTTGGAnn
SEQ ID NO. 129
ggagccaagATACAGTTTGGnn
SEQ ID NO. 130
cggagccaagATACAGTTTGnn
SEQ ID NO. 131
gcggagccaagATACAGTTTnn
SEQ ID NO. 132
tgcggagccaagATACAGTTnn
SEQ ID NO. 133
gtgcggagccaagATACAGTnn
SEQ ID NO. 134
agtgcggagccaagATACAGnn
SEQ ID NO. 135
gagtgcggagccaagATACAnn
SEQ ID NO. 136
tgagtgcggagccaagATACnn
SEQ ID NO. 137
ctgagtgcggagccaagATAnn
SEQ ID NO. 138
gctgagtgcggagccaagATnn
SEQ ID NO. 139
ggctgagtgcggagccaagAnn

In some aspects, the junction in MAN2A1 is set forth in SEQ ID NO: 140 (ccgactataccacatacaggGTACCTTGTGGTCTATAATC with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 141-159:

SEQ ID NO. 141
gGTACCTTGTGGTCTATAATnn
SEQ ID NO. 142
ggGTACCTTGTGGTCTATAAnn
SEQ ID NO. 143
aggGTACCTTGTGGTCTATAnn
SEQ ID NO. 144
caggGTACCTTGTGGTCTATnn
SEQ ID NO. 145
acaggGTACCTTGTGGTCTAnn
SEQ ID NO. 146
tacaggGTACCTTGTGGTCTnn
SEQ ID NO. 147
atacaggGTACCTTGTGGTCnn
SEQ ID NO. 148
catacaggGTACCTTGTGGTnn
SEQ ID NO. 149
acatacaggGTACCTTGTGGnn
SEQ ID NO. 150
cacatacaggGTACCTTGTGnn
SEQ ID NO. 151
ccacatacaggGTACCTTGTnn
SEQ ID NO. 152
accacatacaggGTACCTTGnn
SEQ ID NO. 153
taccacatacaggGTACCTTnn
SEQ ID NO. 154
ataccacatacaggGTACCTnn
SEQ ID NO. 155
tataccacatacaggGTACCnn
SEQ ID NO. 156
ctataccacatacaggGTACnn
SEQ ID NO. 157
actataccacatacaggGTAnn
SEQ ID NO. 158
gactataccacatacaggGTnn
SEQ ID NO. 159
cgactataccacatacaggGnn

In some aspects, the present disclosure concerns a splice junction in gene DOCK1. In some aspects the splice junction include the sequence as set forth in SEQ ID NO: 160 (aaaattgattatggaaacagCTTTTTATAACTATGATGCC with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 161-179:

SEQ ID NO. 161
gCTTTTTATAACTATGATGCnn
SEQ ID NO. 162
agCTTTTTATAACTATGATGnn
SEQ ID NO. 163
cagCTTTTTATAACTATGATnn
SEQ ID NO. 164
acagCTTTTTATAACTATGAnn
SEQ ID NO. 165
aacagCTTTTTATAACTATGnn
SEQ ID NO. 166
aaacagCTTTTTATAACTATnn
SEQ ID NO. 167
gaaacagCTTTTTATAACTAnn
SEQ ID NO. 168
ggaaacagCTTTTTATAACTnn
SEQ ID NO. 169
tggaaacagCTTTTTATAACnn
SEQ ID NO. 170
atggaaacagCTTTTTATAAnn
SEQ ID NO. 171
tatggaaacagCTTTTTATAnn
SEQ ID NO. 172
ttatggaaacagCTTTTTATnn
SEQ ID NO. 173
attatggaaacagCTTTTTAnn
SEQ ID NO. 174
gattatggaaacagCTTTTTnn
SEQ ID NO. 175
tgattatggaaacagCTTTTnn
SEQ ID NO. 176
ttgattatggaaacagCTTTnn
SEQ ID NO. 177
attgattatggaaacagCTTnn
SEQ ID NO. 178
aattgattatggaaacagCTnn
SEQ ID NO. 179
aaattgattatggaaacagCnn

In some aspects, the junction in DOCK1 is set forth in SEQ ID NO: 180 (gcgaccttttggagtggctgCTTTTTATAACTATGATGCC with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 181-199:

SEQ ID NO. 181
gCTTTTTATAACTATGATGCnn
SEQ ID NO. 182
tgCTTTTTATAACTATGATGnn
SEQ ID NO. 183
ctgCTTTTTATAACTATGATnn
SEQ ID NO. 184
gctgCTTTTTATAACTATGAnn
SEQ ID NO. 185
ggctgCTTTTTATAACTATGnn
SEQ ID NO. 186
tggctgCTTTTTATAACTATnn
SEQ ID NO. 187
gtggctgCTTTTTATAACTAnn
SEQ ID NO. 188
agtggctgCTTTTTATAACTnn
SEQ ID NO. 189
gagtggctgCTTTTTATAACnn
SEQ ID NO. 190
ggagtggctgCTTTTTATAAnn
SEQ ID NO. 191
tggagtggctgCTTTTTATAnn
SEQ ID NO. 192
ttggagtggctgCTTTTTATnn
SEQ ID NO. 193
tttggagtggctgCTTTTTAnn
SEQ ID NO. 194
ttttggagtggctgCTTTTTnn
SEQ ID NO. 195
cttttggagtggctgCTTTTnn
SEQ ID NO. 196
ccttttggagtggctgCTTTnn
SEQ ID NO. 197
accttttggagtggctgCTTnn
SEQ ID NO. 198
gaccttttggagtggctgCTnn
SEQ ID NO. 199
cgaccttttggagtggctgCnn

In some aspects, the present disclosure concerns a splice junction in gene HOMER1. In some aspects the splice junction include the sequence as set forth in SEQ ID NO: 200 (gcattgccattttcacatagGGAACAACCTATCTTCAGCA with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 201-219:

SEQ ID NO. 201
gGGAACAACCTATCTTCAGCnn
SEQ ID NO. 202
agGGAACAACCTATCTTCAGnn
SEQ ID NO. 203
tagGGAACAACCTATCTTCAnn
SEQ ID NO. 204
atagGGAACAACCTATCTTCnn
SEQ ID NO. 205
catagGGAACAACCTATCTTnn
SEQ ID NO. 206
acatagGGAACAACCTATCTnn
SEQ ID NO. 207
cacatagGGAACAACCTATCnn
SEQ ID NO. 208
tcacatagGGAACAACCTATnn
SEQ ID NO. 209
ttcacatagGGAACAACCTAnn
SEQ ID NO. 210
tttcacatagGGAACAACCTnn
SEQ ID NO. 211
ttttcacatagGGAACAACCnn
SEQ ID NO. 212
attttcacatagGGAACAACnn
SEQ ID NO. 213
cattttcacatagGGAACAAnn
SEQ ID NO. 214
ccattttcacatagGGAACAnn
SEQ ID NO. 215
gccattttcacatagGGAACnn
SEQ ID NO. 216
tgccattttcacatagGGAAnn
SEQ ID NO. 217
ttgccattttcacatagGGAnn
SEQ ID NO. 218
attgccattttcacatagGGnn
SEQ ID NO. 219
cattgccattttcacatagGnn

In some aspects, the present disclosure concerns a splice junction in gene ST18. In some aspects the splice junction include the sequence as set forth in SEQ ID NO: 220 (acatccagcttccacagatgCTTATCTGGATGTCCTCTCA with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 221-239:

SEQ ID NO. 221
gCTTATCTGGATGTCCTCTCnn
SEQ ID NO. 222
tgCTTATCTGGATGTCCTCTnn
SEQ ID NO. 223
atgCTTATCTGGATGTCCTCnn
SEQ ID NO. 224
gatgCTTATCTGGATGTCCTnn
SEQ ID NO. 225
agatgCTTATCTGGATGTCCnn
SEQ ID NO. 226
cagatgCTTATCTGGATGTCnn
SEQ ID NO. 227
acagatgCTTATCTGGATGTnn
SEQ ID NO. 228
cacagatgCTTATCTGGATGnn
SEQ ID NO. 229
ccacagatgCTTATCTGGATnn
SEQ ID NO. 230
tccacagatgCTTATCTGGAnn
SEQ ID NO. 231
ttccacagatgCTTATCTGGnn
SEQ ID NO. 232
cttccacagatgCTTATCTGnn
SEQ ID NO. 233
gcttccacagatgCTTATCTnn
SEQ ID NO. 234
agcttccacagatgCTTATCnn
SEQ ID NO. 235
cagcttccacagatgCTTATnn
SEQ ID NO. 236
ccagcttccacagatgCTTAnn
SEQ ID NO. 237
tccagcttccacagatgCTTnn
SEQ ID NO. 238
atccagcttccacagatgCTnn
SEQ ID NO. 239
catccagcttccacagatgCnn

In some aspects, the present disclosure concerns a splice junction in gene ST18. In some aspects the splice junction include the sequence as set forth in SEQ ID NO: 240 (atttagttgattctctgaagTTGTTGACCTCCTGTACTGG with the junction being the middle point, between small and large caps). Accordingly, a ss or ds RNA may include at least about 20 or 21 nucleotides that span the identified junction. In some aspects, the ss or ds RNA may include a sequence binding to one or more of SEQ ID Nos 241-259:

SEQ ID NO. 241
gTTGTTGACCTCCTGTACTGnn
SEQ ID NO. 242
agTTGTTGACCTCCTGTACTnn
SEQ ID NO. 243
aagTTGTTGACCTCCTGTACnn
SEQ ID NO. 244
gaagTTGTTGACCTCCTGTAnn
SEQ ID NO. 245
tgaagTTGTTGACCTCCTGTnn
SEQ ID NO. 246
ctgaagTTGTTGACCTCCTGnn
SEQ ID NO. 247
tctgaagTTGTTGACCTCCTnn
SEQ ID NO. 248
ctctgaagTTGTTGACCTCCnn
SEQ ID NO. 249
tctctgaagTTGTTGACCTCnn
SEQ ID NO. 250
ttctctgaagTTGTTGACCTnn
SEQ ID NO. 251
attctctgaagTTGTTGACCnn
SEQ ID NO. 252
gattctctgaagTTGTTGACnn
SEQ ID NO. 253
tgattctctgaagTTGTTGAnn
SEQ ID NO. 254
ttgattctctgaagTTGTTGnn
SEQ ID NO. 255
gttgattctctgaagTTGTTnn
SEQ ID NO. 256
agttgattctctgaagTTGTnn
SEQ ID NO. 257
tagttgattctctgaagTTGnn
SEQ ID NO. 258
ttagttgattctctgaagTTnn
SEQ ID NO. 259
tttagttgattctctgaagTnn

### CircRNA Expression

In further aspects, the present disclosure concerns the identification that adenosine deaminase enzymes ADARs (adenosine deaminases acting on RNAs) can generally lead to the translation of circular RNA molecules, including both the 12->7 and 12->10 circRNAs disclosed herein. However, as further set forth herein, the ADAR-based translation of circRNAs is not limited to just these circRNAs, but provides a general mechanism that allows for the translation and/or expression of circRNAs. In some aspects the cirRNAs include an ATG start motif. In other aspects, the circRNAs do not require the inclusion of a circRNA ATG start motif in order for translation and/or expression of a peptide(s) encoded by the circRNA. In some aspects, ADARs may convert adenosines in a circRNA to inosine residues, which are deaminated adenosines. In some aspects, the conversion of one or more adenosines to inosines allows one or more adenosines within the circRNA to appear as a guanosine to the translation machinery within a cell. In other aspects, the conversion of one or more adenosines within the circRNA to inosine allows for the inosine to base-pair with cytosine, guanine, and/or uracil. In some aspects, the ADARs bind and/or recognize double-stranded regions within the circRNA that allows for the ADARs to deaminate one or more adenosine residues within the circRNA to inosine.

In some aspects, the present disclosure concerns the identification that one or more ADAR enzymes, such as human ADAR1 and/or ADAR2 will recognize, bind and translate and/or express one or more peptide sequences from a circRNA sequence. The ADAR enzymes can be of an eukaryotic organism, including invertebrates, vertebrates, fish, birds, mammals, reptiles, amphibians, nematodes, protozoa, porifera, coelenterate, Platyhelminthes, annelids, echinoderms, molluscs, and arthropods. In some aspects, an ADAR enzyme, such as ADAR1 and/or ADAR2, binds or interacts with a circRNA and causes the translation thereof. In some aspects, the ADAR enzyme may recognize a short double-stranded region (5-10 nt double strand with interruptions) within a circRNA from complementary regions between the 5′ direction and the reverse 3′ direction.

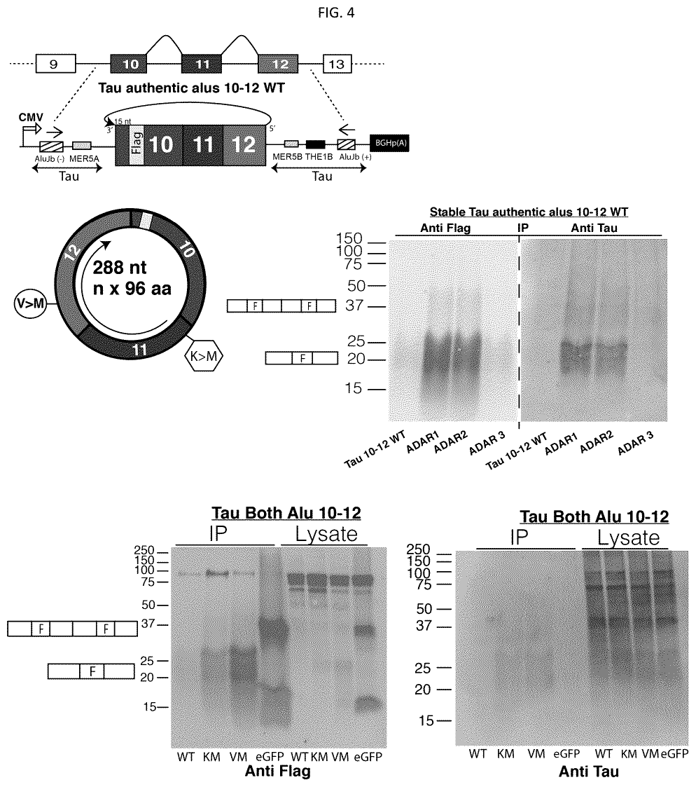

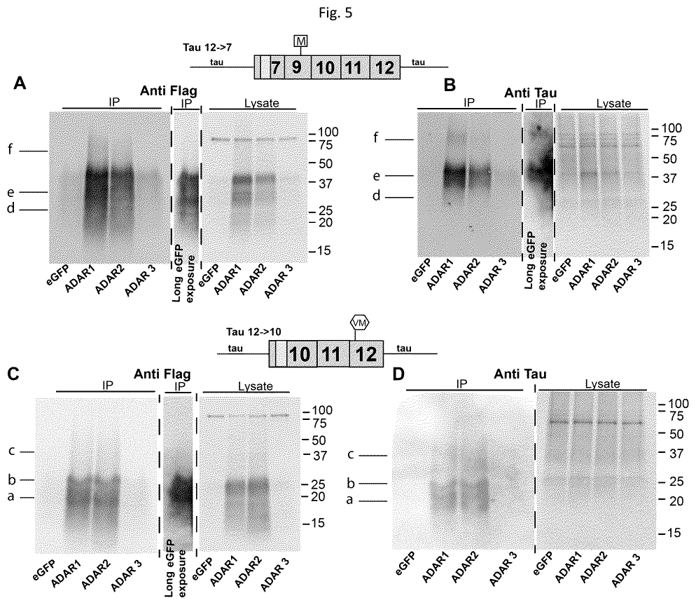

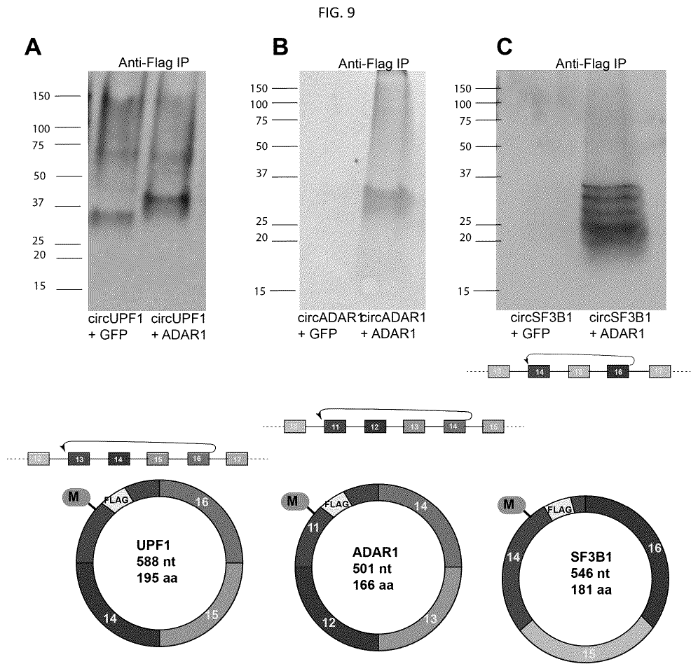

As set forth in the working examples herein, introducing active ADAR enzymes allowed for the translation and/or expression of protein products encoded by circRNAs, not only for the two tau circRNAs (FIGS. 4 and 5), but for circRNAs in general (FIG. 9). Further, the working examples set forth evidence that inhibition of ADAR enzymes is caused by the presence of AKT and/or the kinase activity thereof. In some aspects, ADAR RNA editing of circRNAs is inhibited by phosphorylation from an AKT kinase.

In some aspects, the present disclosure therefore concerns methods to regulate translation and/or expression of circRNAs by introducing ADAR enzymes or by introducing AKT kinases. ADAR activity is decreased by 8-azaadenosine and 8-chloroadenosine, however these compounds show limited selectivity.

In some aspects, regulating can be increased activity. In other aspects, regulating can be decreased or inhibited activity. In some aspects, the methods may include introducing an agonist or an antagonist to an ADAR enzyme and/or to an AKT kinase. In some aspects, the methods may include administration of MK-2206, SC79, capivasertib, ipatasertib, honokiol, A-674563, TAS-117, 3,4,5-tricaffeoylquinic acid, GSK-690693, AKT inhibitor VIII, artemisinin, perifosine, oridonin, guggulsterone, miransertib, triciribine, afuresertib. uprosertib, recilisib, SU6656, A-443654, scutellarin, isobavachalcone, deguelin, miltefosine, alpha-linolenic acid, YS-49, LM22B-10, fumonisin B1, and similar. In some aspects, the methods may include over-expressing a constitutively active ADAR enzyme or AKT kinase. In some aspects, the methods may include over-expressing an inactive ADAR, such as ADAR 3 and/or a phosphorylated ADAR1 and/or phosphorylated ADAR2. In some aspects, a phosphomimetic may employed wherein one or more serine(s) and/or threonine(s) can be mutated to a glutamate residue and/or an aspartate residue and/or an alanine residue. For example, over-expression of a phosphomimetic of ADAR1 and/or ADAR2 may down-regulate circRNA translation and/or expression. Methods may include the introduction and/or use of small molecule compounds, viral expression systems, siRNA, lipid delivery systems, antibodies or antibody fragments, and other techniques know to those skilled in the art. The methods may be performed in vitro, in vivo, and/or ex situ.

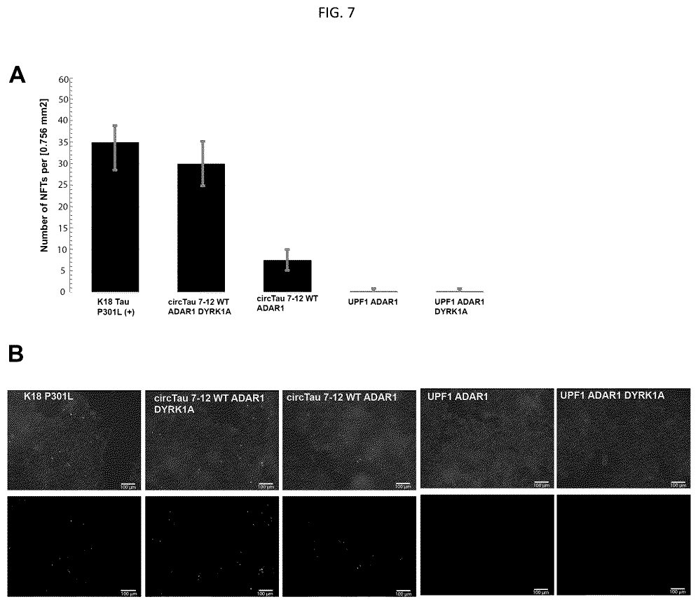

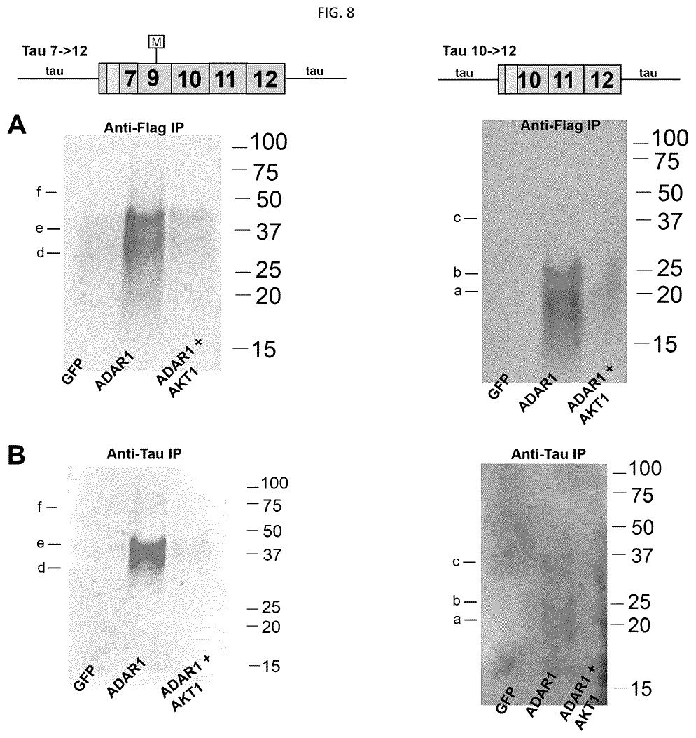

It will be apparent to those in the art that in some instances it may be of benefit or potential benefit to increase or stimulate the expression of peptides from a circRNA. In other aspects, it may be of benefit or potential benefit to reduce or inhibit expression of peptides from a circRNA. For example, as identified herein, expression of circRNA proteins can cause neurofibrillary tangle (NFT) accumulation (see FIG. 7A). Similarly, abnormal circRNAs are currently linked to a number of cancers (see, e.g. Liu et al. Front. Oncol. 20 May 2020, doi.org/10.3389/fonc.2020.00663).

### Tau CircRNA Expression

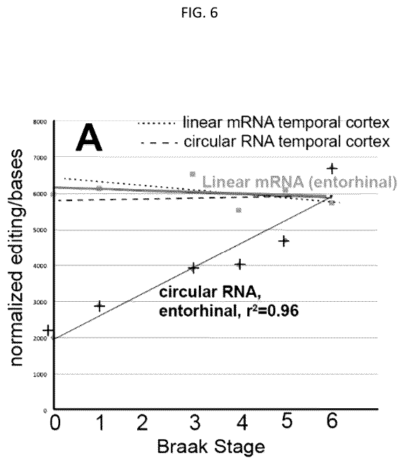

In further aspects, the present disclosure concerns the identification that the ADAR-dependent expression of one or more peptides from the tau backspliced circRNA can cause NFT accumulation (see FIG. 7). These data coupled with the additional observation that Adenosine to in inosine editing in entorhinal cortex circRNA generally increase as Braak stages increase, (see FIG. 6) suggest a significant role of the ADAR editing of tau circRNA in Alzheimer's disease. Accordingly, methods to disrupt the RNA editing can provide a treatment to alleviate the onset or advancing of Alzheimer's disease.

### Additional CircRNA Peptides

In some aspects, the present disclosure concerns expressed protein and/or peptide product from additional circRNAs created by backsplicing. In some aspects, the backsplicing can allow for a frameshift or alternative ATG start cite to be used for translation. In some aspects, the splicing can append a different or additional sequence to a known protein. In some aspects, the splicing can lead to a continuous repeat of an amino acid sequence, such as when the circRNA possesses a multiple of three nucleotides, thereby allowing the translation machinery to repeat the same translation.

In some aspects, the present disclosure concerns novel protein products from circRNAs. In some aspects, the circRNAs are associated with or correlate to the onset and/or progression of Alzheimer's disease. In some aspects, the circRNAs include circRNAs produced by the genes MAN2A1, HOMER1, DOCK1, ST18, and RTN4 (see, Dube et al. Nature neuroscience 22:1903-1912, 2019).

In some aspects, the present disclosure concerns one or more peptides expressed from alternative splicing of MAN2A1. SEQ ID Nos: 80, 100, 120, and 140. In some aspects the circRNA of MAN2A1 at the splice junction set forth in SEQ ID NO: 80 may produce of one or more peptides comprising or consisting of: IPITTQGQLSMLQ (SEQ ID NO: 260), MRSSQILETQSSI (SEQ ID NO: 261), VEFGSKDLTLLMNLMNGTLNPFKSLWCLIPITTQ (SEQ ID NO: 262) and/or MVRKVHKAISAKVLAHIFCPHNYPSQLTLQTVCLLHKVEVTIQMCRWWSLEARI (SEQ ID NO: 263). In some aspects, the expressed protein or peptide may include one or more of the peptides of: MLQEKIDHLERLLAENNEIISNIRDSVINLSESVEDGPKSSQSNFSQGAGSHLLPSQLSLSV DTADCLFASQSGSHNSDVQMVEFGSKDLTLLMNLMNGTLNPFKSLWCLIPITTQ (SEQ ID NO: 264); and/or MLQEKIDHLERLLAENNEIISNIRDSVINLSESVEDGPKSSQSNFSQGAGSHLLPSQLSLSV DTADCLFASQSGSHNSDVQMVEFGSKDLTLLMNLMNGTLNPFKSLWCLIPITTQGQLS MLQEKIDHLERLLAENNEIISNIRDSVINLSESVEDGPKSSQSNFSQGAGSHLLPSQLSLSV DTADCLFASQSGSHNSDVQMVEFGSKDLTLLMNLMNGTLNPFKSLWCLIPITTQ (SEQ ID NO: 265) or multimers thereof.

In some aspects the circRNA of MAN2A1 at the splice junction set forth in SEQ ID NO: 80 may produce of one or more peptides comprising or consisting of: GPALNVARKNRPFGAFAS (SEQ ID NO: 266) and/or MVRKVHKAISAKVLAHIFCPHNYPSQLTLQTVCLLHKVEVTIQMCRCWMFTV (SEQ ID NO: 267) and/or MLQEKIDHLERLLAENNEIISNIRDSVINLSESVEDGPKSSQSNFSQGAGSHLLPSQLSLSV DTADCLFASQSGSHNSDVQMLDVYSLISFDNPDGGVWKQGFDITYESNEWDTEPLQVF VVPHSHNDPGPALNVARKNRPFGAFAS (SEQ ID NO: 268).

In some aspects the circRNA of MAN2A1 at the splice junction set forth in SEQ ID NO: 100 may produce of one or more peptides comprising or consisting of: MVRKVHKAISAKVLAHIFCPHNYPSQLTLQTVCLLHKVEVTIQMCRCWMFTV (SEQ ID NO: 269), MVEFGSKDLTLLMNLMNGTLNPFKLVEDFQ (SEQ ID NO: 270); MVLKLKEDSRRKFIWSEISYLSKWWDIIDIQKKDAVKRASSQCCKKK (SEQ ID NO: 271), and/or MLQEKIDHLERLLAENNEIISNIRDSVINLSESVEDGPKSSQSNFSQGAGSHLLPSQLSLSV DTADCLFASQSGSHNSDVQMLDVYSLISFDNPDGGVWKQGFDITYESNEWDTEPLQVG (SEQ ID NO: 272).

In some aspects the circRNA of MAN2A1 at the splice junction set forth in SEQ ID NO: 120 may produce of one or more peptides comprising or consisting of: MRWIKQMKLRETRANRCSLF (SEQ ID NO: 273), MPIEMIITGVAILHPDPFTNEWTESWNLI (SEQ ID NO: 274), RYSLELYQIFLMRWIKQMKLRETRANRCSLF (SEQ ID NO: 275), and/or MFPVLSGDFFTYADRDDHYWSGYFTSRPFYKRMDRIMESHLRLFHSLMVLEKIIGNSAF LLILKDKLTYDSYSPDTFLEMDLKQKSQDSLPQKNIIRLSAEPRYSLELYQIFLMRWIKQ MKLRETRANRCSLF (SEQ ID NO: 276).

In some aspects the circRNA of MAN2A1 at the splice junction set forth in SEQ ID NO: 140 may produce of one or more peptides comprising or consisting of: VPCGL (SEQ ID NO: 277), MLLIESDYTTYRVPCGL (SEQ ID NO: 278), and/or MSLMFTQHRPLSE (SEQ ID NO: 279).

In some aspects the circRNA of DOCK11 at the splice junction set forth in SEQ ID NO: 160 may produce of one or more peptides comprising or consisting of: MFRSVRHMIYDLIEWRSQILSGTLPQDELKELKKKVTAKIDYGN (SEQ ID NO: 280), MFRSVRHMIYDLIEWRSQILSGTLPQDELKELKKKVTAKIDYGNSFL (SEQ ID NO: 281), METAFYNYDARGADELSLQIGDTVHILETYEGWYRGYTLRKKSKKGIFPASYIHLKEAI VEGKGQHETVIPGDLPLIQEVTTTLREWSTIWRQLYVQDNREMFRSVRHMIYDLIEWRS QILSGTLPQDELKELKKKVTAKIDYGNSFL (SEQ ID NO: 282), MAITNSFWNSASG (SEQ ID NO: 283), MMPEERMNFLYRSETLCTS (SEQ ID NO: 284), MKGGTEVTRYEKSLRRVYFLLHIFILKKR (SEQ ID NO: 285), MKQSSRVTSPSSRKSPRHSESGPPSGGSSTCKITGRCFEVCGT (SEQ ID NO: 286), and/or MTLLNGDHKFFLELCLRMNSKN (SEQ ID NO: 287).

In some aspects the circRNA of DOCK11 at the splice junction set forth in SEQ ID NO: 180 may produce of one or more peptides comprising or consisting of: MELRDNNTRKLTSGLRRPFGVAAFYNYDARGADELSLQIGDTVHILETYEGWYRGYTL RKKSKKDLGSKDLKREKISFVCQIVRVGRMELRDNNTRKLTSGLRRPFGVA (SEQ ID NO: 288), MMPEERMNFLYRSETLCT (SEQ ID NO: 289), FGVAAFYNYD (SEQ ID NO: 290) and/or MKGGTEVTRYEKSLRRTSEAKT (SEQ ID NO: 291).

In some aspects the circRNA of HOMER1 at the splice junction set forth in SEQ ID NO: 200 may produce of one or more peptides comprising or consisting of: MTFTKTSQKFGQWADSRANTVYGLGFSSEHHLSKFAEKFQEFKEAARLAKEKSQEKME LTSTPSQESAGGDLQSPLTPESINGTDDERTPDVTQNSEPRAEPTQNALPFSHREQPIFSTR AHVFQIDPNTKKNWVPTSKHAVTVSYFYDSTRNVYRIISLDGSKAIINSTITPNMTFTKTS QKFGQWADSRANTVYGLGFSSEHHLSKFAEKFQEFKEAARLAKEKSQEKMELTSTPSQ ESAGGDLQSPLTPESINGTDDERTPDVTQNSEPRAEPTQNALPFSH (SEQ ID NO: 292), LPFSHREQPIFS (SEQ ID NO: 293) MHCHFHIGNNLSSALELMSSKLTQTQRRTGYPPASMQLLCLISMTAQEMCIG (SEQ ID NO: 294), MSSKLTQTQRRTGYPPASMQLLCLISMTAQEMCIG (SEQ ID NO: 295), and/or MDWDSPLSIIFRNLQKSFRNLKKLLD (SEQ ID NO: 296).

In some aspects the circRNA of ST18 at the splice junction set forth in SEQ ID NO: 220 may produce of one or more peptides comprising or consisting of: MTIKLKATGDHIYGEQLKDDRGGEQTHRTEQ (SEQ ID NO: 297), MESNLKTIEEENKLIEQNNESLLKELAGLSQALISSLADIQLPQMLIWMSSQCTSYQKGQ GF (SEQ ID NO: 298), and/or MLIWMSSQCTSYQKGQGF (SEQ ID NO: 299).

In some aspects the circRNA of RTN4 at the splice junction set forth in SEQ ID NO: 180 may produce of one or more peptides comprising or consisting of: MYKGVIQAIQKSDEGHPFRAYLESEVAISEELVQKYSNSALGHVNCTIKELRRLFLVDD LVDSLKLLTSCTGETLRRLEWCLVPAYSCCF (SEQ ID NO: 300), LLTSCTGETLRRLEWCLVPAYSCC (SEQ ID NO: 301), and/or MKATHSGHIWNLKLLYLRSWFRSTVILLLVM (SEQ ID NO: 302).

The splice variants of MAN2A1, HOMER1, DOCK1, ST18, and RTN4 are set forth in SEQ ID Nos: 303-312).

### Antibodies

In some aspects, the present disclosure concerns antibodies or fragments thereof that bind to the peptides produced by the circRNAs as set forth herein. In some aspects, the antibodies bind to a peptide as set forth in one of SEQ ID NOs: 260-302. It will be appreciated that the antibody may be a monoclonal or a polyclonal antibody. The antibody may be of an immunoglobulin (Ig) selected from IgG, IgA, IgM, and/or IgE. The antibody may a single heavy chain or of a heavy and light chain. Fragments may include portions of the antibody that retain the ability to bind the circRNA peptides, including three complementary-determining regions (CDRs) within a heavy chain or six CDRs shared between heavy and light chains. Fragments may include VH domains, VL domains, VHH domains, V-NAR domains, V domains, Fab fragments, Fab₂ fragments, scFv fragments, diabodies, miniboides, tribodies, tetrabodies, and the like understood in the art.

### Methods of Treatment

In some aspects, the present disclosure concerns identifying the presence of the circRNAs as set forth herein and/or the proteins or peptides correlates to the onset and/or progression of Alzheimer's. In some aspects, the methods of the present disclosure concern detecting or assaying for the presence of one or more of the circRNAs and/or proteins or peptides expressed therefrom. In some aspects, the methods include obtaining a sample from the subject. In some aspects, the sample is of a fluid from the subject, such as cerebrospinal fluid and/or blood or plasma. In some aspects, the methods include assaying the sample for the presence of one or more of the circRNAs as set forth herein, including detecting the nucleotide and/or expressed protein or peptide. In some aspects, the methods include contacting or administering to the sample one or more of the nucleotides (such as one or more of SEQ ID Nos: 1-259) disclosed herein to assay for hybridization to a nucleotide in the sample. In some aspects, the methods include contacting or administering one or more of the antibodies as described herein with the sample to assay for binding between the antibody and an expressed protein or peptide in the sample. Such may include contacting and/or administering to the sample an antibody that binds one of SEQ ID Nos: 260-302. Determining whether a nucleotide hybridizes or binds to a nucleotide sequence in the sample or determining whether an antibody binds to an antigenic protein or peptide within the sample may require secondary or additional reagents to confirm the reaction, as are understood in the art with techniques such as Northern blotting, Southern blotting, Western blotting, immunoprecipitation, immunostaining, ELISA, colorometric responses, fluorescent response, radiolabeling, conjugated antibodies, labeled antibodies, labeled nucleotides, and the like.

In some aspects, the determining of the presence of one or more of the circRNAs and/or a peptide or protein expressed therefrom as set forth herein is indicative of the onset or progression of Alzheimer's disease and/or neurodegeneration in the subject. In some aspects, such results may allow for a course of treatment for the subject. In some aspects, such treatment may include administration to the subject of one or more siRNA, dsRNA or antisense thereof as described herein to reduce or lower one or more circRNA and/or protein/peptide expressed thereby. In some aspects, the methods may include assaying the subject over a period of time. As described herein, circRNA levels and/or proteins/peptides expressed thereby can correlate to disease progression. In some aspects, one may determine the Braak stage of the patient by determining the concentration and/or amount of circRNA and/or expressed protein/peptide thereof in the subject or a sample obtained therefrom. In some aspects, a Braak stage may be determined from one assayed sample. For example, protein products made from circMAPT, circMAN2A1, circHOMER1, circST18 and circRTN4 can be extracted from up to 100 ml of cerebrospinal fluid. Antibodies specific to peptides encoded by circRNAs, but not linear RNAs will be coupled to a solid phase (separose or IgG-coated magnetic beads) and this affinity matrix will purify the circRNA peptides from cerebrospinal fluid. The purified protein will be detected in a Western Blot or Elisa format. In further aspects, a Braak stage may be determined over a period of time wherein two or more samples are assayed at two or more different time points. It will be apparent that such time points should be adequately spaced in time, such as by one week or more, including up to one month, two months, and six months, up to several years. In some aspects, monitoring and or detecting an increase in circRNA and/or proteins/peptides expressed thereby over a period of time will allow one to assess the rate of disease or neurodegradation in part based on the change in concentration in consideration of the time elapsed between assays.

Further aspects and advantages of this disclosure are provided in the following section, which should be considered as illustrative only.

### EXAMPLES

To test the efficacy of the siRNA compositions, cells were established that expressed a FLAG epitope in exon 10, thereby allowing for both immunoprecipitation of any expressed circRNA construct and for immunodetection thereof in both lysate fractions and immunoprecipitates thereof. See FIG. 4.

Silencing the 12->10 circRNA:

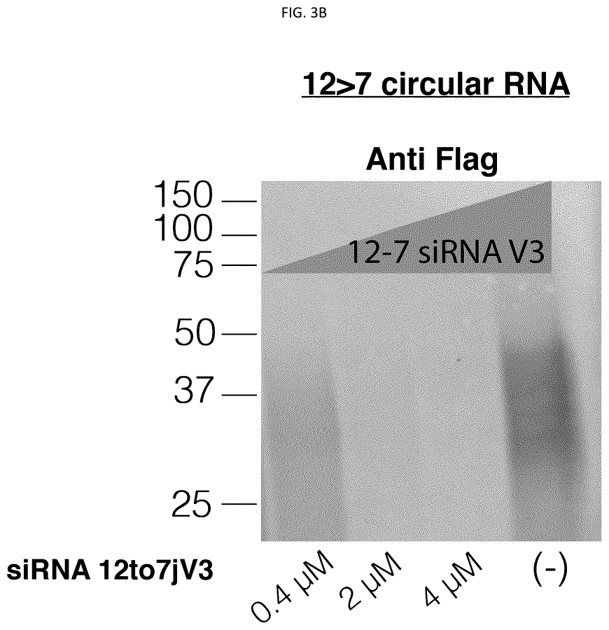

Cells were allowed to undergo one and two rounds of translation of the 12->10 circRNA as verified by an anti-tau antiserum. Different concentrations of siRNA (sense: 5′AUAAAAAGGUGCAGAUAAUtt (SEQ ID NO: 9 with 3′ thymine), antisense: 3′dTTUAUUUUUCCACGUCUAUUA (SEQ ID NO: 27 with 3′ thymine)) were transfected in a volume of 150 μl Opti-MEM (Invitrogen) with 10 μl lipofectamine 2000 (Invitrogen). The transfection mixture (320 μl) was added to cells plated in a 6-well plate with the media removed in allowed to incubate for 20 min at 37° C. under 5% CO₂. 2 mL of fresh media supplemented with 1 μL/mL hygromycin was then added and the cells further incubated for 24 hrs. After the initial 24 hours, cell media was replaced and the cells were then further incubated for an additional 48 hrs. Cells were then lysed and immunoprecipitated (IP) using an M2 FLAG Ab with a normalized protein concentration of ˜200 μg. Following the IP, the proteins were denatured, resolved by electrophoresis on an acrylamide gel and transferred to a membrane for Western blotting. The membrane was probed with anti-tau antibodies and anti-FLAG antibodies. FIG. 3A shows a representative blot, where the introduction of the siRNA prevented the formation of tau.

### Silencing the 12->7 circRNA

As with above, cells were allowed to undergo translation and incubated with 12->7 jv2 or jv3 (SEQ ID NOS: 51 and 69 and SEQ ID NOS: 53 and 71, respectively). Following the same protocol as above, 200 μg of total protein was immunoprecipitated as above and ultimately Western blotted for tau. The total volume of lysate following the IP was about 180 μl, so 3 wells were combined from a 6-well plate for each sample to increase protein concentration. FIG. 3B shows the silencing of the 12->7 circRNA with lane 1 being 1 μl of jv2 in 540 μg total protein, lane 2 being 5 μl of jv2 in 532 μg total protein and lane 3 being 10 μl of jv2 in 480 μg total protein. Lane 4 is a negative control of WT 12->7 in 488 μg total protein. Lanes 5-8 further examined jv3 silencing of the 12->7 circRNA, with lane 5 being 1 μl of jv3 in 468 μg total protein, lane 6 being 5 μl of jv3 in 508 μg total protein and lane 7 being 10 μl of jv3 in 304 μg total protein. Lane 8 was a further negative control of WT 12->7 in 420 μg total protein.

### Expression of the tau circRNAs

It was next observed that RNA editing caused by overexpression of the human proteins ADAR1 and ADAR2 strongly promotes translation of both the 12->7 and 12->10 circRNAs. The protein resulting from the 12->7 backsplice is likely present in most brain parts. The protein made from 12->10 is limited to expression only when the identified mutations are present or when A->I RNA editing occurs. Constructs introducing a FLAG insert in the circRNA were utilized to provide an epitope for purification and immunoprobing.

FIG. 4 shows that cotransfection with ADAR1 and ADAR2 dramatically increases translation of the 12->10 backsplice RNA despite the absence of the start-codon in the wild-type (WT) circRNA. ADAR1 and ADAR2 catalyze the conversion of Adenosines to Inosines which can be read as guanosines. ADAR3 encodes a related human enzyme that however lacks the catalytic Adenosine to inosine activity. FIG. 5 shows similar ADAR1 and ADAR2 dependent translation and/or expression of FLAG-circRNA of 12->7.

### Tau circRNA Expression in AD

After identifying that the tau circRNAs can be expressed as peptides, it was evaluated for any potential role or correlation the circRNAs may have with regard to the onset or progression of tau neurofibrillary tangles (NFTs). Samples from the temporal cortex and entorhinal cortex were assayed for linear and circRNA during the six Braak stages. A correlation of Braak stage (AD severity) with adenosine to inosine editing of all circular RNA in entorhinal cortex was observed (FIG. 6). Thus A>I editing could be the trigger that starts AD development through tau circular RNAs. There is no change in temporal cortex, but AD starts in entorhinal cortex.

It was next assessed whether the protein(s) or peptide(s) produced by the circRNA cause neurofibrillary fibril formation. A>I RNA editing not only increases protein production, it changes amino acid usage, for example an AUA codon (Ile) is edited into an AUI codon which will be read as Methionine. The protein translated from ADAR1 edited circular Tau RNAs was accordingly used in biosensor cells assays that indicate neurofibrillary tangles as bright ‘green dots’.

The protein generated from tau 12->7 WT co-transfected with ADAR1 was purified and then added to cells. The resulting pathology was indicated in the Biosensor cells (FIG. 7A, B). The positive control, K18 is a synthetic peptide that consist of the microtubule binding domains of the linear tau protein containing the FTDP-17 mutation P301L that promotes tau aggregation and pathology (FIG. 7B) and negative control mock transfection.

### ADAR Enzyme Activity on Other CircRNAs

It was next examined whether ADAR enzymes effectuate the translation and/or expression of other circRNAs. FLAG motifs were inserted into the circRNAs of UPF1, ADAR1 and SF3B1 and then cotransfected with either GFP (control) or ADAR1 to determine whether a protein product is expressed. As set forth in FIG. 9, ADAR1 produced expression of FLAG-tagged proteins in all instances. This suggests a role for the circRNA forming a partial double-stranded complex that is recognized by ADAR enzymes and allows for RNA editing. The RNA editing then allows for the effective translation of the circRNA.

### CircRNA Expression

Entorhinal cortex was removed between 1-3 hrs post mortem. One sample for each Braak stage was used. RNA from this tissue was isolated using Qiagen columns and subjected to RNAseq. For the RNAseq, ribosomal RNA was removed (ribo minus kit, Invitrogen) and linear RNA was removed through RNAse R treatment. Priming was with Illumina random hexamers. Circular RNAs were identified using their backsplice junctions and assembled from the overlapping reads. The mean expression of the circRNAs was normalized to all reads of linear RNAs from the same sample and expressed as the fraction of junction reads.

### Aspects

A first aspect, either alone or in combination with any other aspect described herein concerns an isolated double stranded (ds) silencing ribonucleic acid (siRNA) to silence expression of a backsplice circular RNA (circRNA) from exon 12 of the MAPT gene comprising a fragment of exon 12 and a fragment of the backspliced exon.

A second aspect, either alone or in combination with any other aspect described herein concerns an isolated ds siRNA comprising between 17 and 22 contiguous nucleic acids from SEQ ID NO: 1 and/or SEQ ID NO: 39.

A third aspect, either alone or in combination with any other aspect described herein concerns the isolated ds siRNA of the second aspect, wherein the contiguous nucleic acids comprise at least one nucleic acid from exon 12 and one nucleic acid from either exon 10 or exon 7.

A fourth aspect, either alone or in combination with any other aspect described herein concerns an isolated ds siRNA comprising a nucleic acid sequence selected from the group consisting of SEQ ID NOs: 2-19.

A fifth aspect, either alone or in combination with any other aspect described herein concerns an isolated ds siRNA comprising a nucleic acid sequence selected from the group consisting of SEQ ID NOs: 40-57.

A sixth aspect, either alone or in combination with any other aspect described herein concerns the isolated ds siRNA as set forth in any aspect herein, wherein one or more nucleic acids are modified.

A seventh aspect, either alone or in combination with any other aspect described herein concerns the isolated ds siRNA of any preceding aspect, wherein the double strands are two separate annealed strands.

An eighth aspect, either alone or in combination with any other aspect described herein concerns the isolated ds siRNA of any of aspects one through six, wherein the double strands are a self-annealed single strand.

A ninth aspect, either alone or in combination with any other aspect described herein concerns a method of treating or alleviating formation of neurofibrillary tangles comprising administering an isolated ds siRNA as claimed herein.

A tenth aspect, either alone or in combination with any other aspect described herein concerns a method to treat frontotemporal degeneration with Parkinsonism linked to chromosome 17 (FTDP17), comprising administering an isolated ds siRNA as claimed herein.

An eleventh aspect, either alone or in combination with any other aspect described herein concerns the method of the ninth or tenth aspects, wherein the ds siRNA is administered by intrathecal injection.

A twelfth aspect, either alone or in combination with any other aspect described herein concerns a method for increasing translation of a circular RNA molecule comprising administering to the circular RNA an adenosine deaminase acting on RNA (ADAR) activity wherein the ADAR edits at least one adenosine in the circular RNA molecule to an inosine.

A thirteenth aspect, either alone or in combination with any other aspect described herein concerns the method of the twelfth aspect, wherein the circular RNA molecule is a tau circular RNA as described herein.

A fourteenth aspect, either alone or in combination with any other aspect described herein concerns a method for inhibiting translation of a circular RNA molecule in a cell comprising inhibiting an ADAR enzyme within the cell from editing the circular RNA molecule.

A fifteenth aspect, either alone or in combination with any other aspect described herein concerns the method of the fourteenth aspect, wherein the ADAR enzyme is inhibited by phosphorylation of at least one serine or threonine therein.

A sixteenth aspect, either alone or in combination with any other aspect described herein concerns the method of the fifteenth aspect, wherein the ADAR enzyme is phosphorylated by AKT.

A seventeenth aspect, either alone or in combination with any other aspect described herein concerns an isolated nucleic acid that corresponds to a circular RNA (circRNA) sequence comprising a nucleotide sequence of at least twenty contiguous nucleotides, wherein at least a terminal of the contiguous nucleotide sequence corresponds to a first exon within a gene and a second terminus of the contiguous nucleotide sequence corresponds to a second exon within the gene, the first and second exons being different.

An eighteenth aspect, either alone or in combination with any other aspect described herein concerns the isolated nucleic acid of the seventeenth aspect, wherein the contiguous nucleotide sequence is derived from a splice junction site selected from the group consisting of SEQ ID NO: 80, SEQ ID NO: 100, SEQ ID NO: 120, SEQ ID NO: 140, SEQ ID NO: 160, SEQ ID NO: 180, SEQ ID NO: 200, SEQ ID NO: 220, and SEQ ID NO: 240.

A nineteenth aspect, either alone or in combination with any other aspect described herein concerns the isolated nucleic acid of the seventeenth or eighteenth aspect, wherein the contiguous nucleotide sequence is comprised of a sequence selected from SEQ ID Nos: 60-259.

A twentieth aspect, either alone or in combination with any other aspect described herein concerns the isolated nucleic acid of aspects seventeen through nineteen, further comprising a complementary strand.

A twenty-first aspect either alone or in combination with any other aspect described herein concerns the isolated nucleic acid of the twentieth aspect, wherein the complementary strand is a separate annealed strand.

A twenty-second aspect, either alone or in combination with any other aspect described herein concerns the isolated nucleic acid of the twentieth aspect, wherein the complementary strand is connected to the contiguous nucleotide sequence to form a self-annealed single strand.

A twenty-third aspect, either alone or in combination with any other aspect described herein concerns the isolated nucleic acid of any of aspects seventeen through twenty, wherein one or more nucleic acids are modified.

A twenty-fourth aspect, either alone or in combination with any other aspect described herein concerns a method for treating or alleviating neurodegeneration in a subject comprising administering the isolated nucleic acid of any of aspects seventeen through twenty-one to the subject.

A twenty-fifth aspect, either alone or in combination with any other aspect described herein concerns an isolated peptide comprising an amino acid sequence selected from the group consisting of SEQ ID NOS: 260-302.

A twenty-sixth aspect, either alone or in combination with any other aspect described herein concerns a nucleotide encoding the isolated peptide of the twenty-fifth aspect.

A twenty-seventh aspect, either alone or in combination with any other aspect described herein concerns an antibody comprising complementary determination regions that bind the isolated peptide of the twenty-fifth aspect.

A twenty-eighth aspect, either alone or in combination with any other aspect described herein concerns the antibody of the twenty-seventh aspect, wherein the antibody is a monoclonal antibody.

A twenty-ninth aspect, either alone or in combination with any other aspect described herein concerns the antibody of the twenty-seventh aspect, wherein the antibody is a polyclonal antibody.

A thirtieth aspect, either alone or in combination with any other aspect described herein concerns a method of determining the onset and/or progression of Alzheimer's disease in a subject, comprising obtaining a sample from the subject and administering to the sample the antibody of the twenty-seventh through twenty-eighth aspect.

A thirty-first aspect, either alone or in combination with any other aspect described herein concerns the method of the thirtieth aspect, further comprising initiating treatment upon determining when the antibody binds an antigen in the sample.

A thirty-second aspect, either alone or in combination with any other aspect described herein concerns the method of the thirtieth aspect, further comprising determining the concentration of antigen in the sample and obtaining a Braak score therefrom.

A thirty-third aspect, either alone or in combination with any other aspect described herein concerns a method for measuring a Braak score in a subject comprising obtaining a sample from the subject; administering to the sample the antibody of the twenty-seventh aspect; and, determining the concentration of antibody binding within the sample, wherein higher antibody binding increases the Braak score.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The drawings are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

We claim:

1. An isolated double stranded (ds) silencing ribonucleic acid (siRNA) to silence expression of a backsplice circular RNA (circRNA) from exon 12 of the MAPT gene comprising a fragment of exon 12 and a fragment of the backspliced exon.

2. An isolated ds siRNA comprising between 17 and 22 contiguous nucleic acids from SEQ ID NO: 1 and/or SEQ ID NO: 39.

3. The isolated ds siRNA of claim 2, wherein the contiguous nucleic acids comprise at least one nucleic acid from exon 12 and one nucleic acid from either exon 10 or exon 7.

4. An isolated ds siRNA comprising a nucleic acid sequence selected from the group consisting of SEQ ID NOs: 2-19.

5. An isolated ds siRNA comprising a nucleic acid sequence selected from the group consisting of SEQ ID NOs: 40-57.

6. The isolated ds siRNA as set forth in prior claim, wherein one or more nucleic acids are modified.

7. The isolated ds siRNA of any preceding claim, wherein the double strands are two separate annealed strands.

8. The isolated ds siRNA of any of claims 1-6, wherein the double strands are a self-annealed single strand.

9. A method of treating or alleviating formation of neurofibrillary tangles comprising administering an isolated ds siRNA as claimed herein.

10. A method to treat frontotemporal degeneration with Parkinsonism linked to chromosome 17 (FTDP17), comprising administering an isolated ds siRNA as claimed herein.

11. The method of claim 9 or 10, wherein the ds siRNA is administered by intrathecal injection.

12. A method for increasing translation of a circular RNA molecule comprising administering to the circular RNA an adenosine deaminase acting on RNA (ADAR) activity wherein the ADAR edits at least one adenosine in the circular RNA molecule to an inosine.

13. The method of claim 12, wherein the circular RNA molecule is a tau circular RNA as described herein.

14. A method for inhibiting translation of a circular RNA molecule in a cell comprising inhibiting an ADAR enzyme within the cell from editing the circular RNA molecule.

15. The method of claim 14, wherein the ADAR enzyme is inhibited by phosphorylation of at least one serine or threonine therein.

16. The method of claim 15, wherein the ADAR enzyme is phosphorylated by AKT.

17. An isolated nucleic acid that corresponds to a circular RNA (circRNA) sequence comprising a nucleotide sequence of at least twenty contiguous nucleotides, wherein at least a terminal of the contiguous nucleotide sequence corresponds to a first exon within a gene and a second terminus of the contiguous nucleotide sequence corresponds to a second exon within the gene, the first and second exons being different.

18. The isolated nucleic acid of claim 17, wherein the contiguous nucleotide sequence is derived from a splice junction site selected from the group consisting of SEQ ID NO: 80, SEQ ID NO: 100, SEQ ID NO: 120, SEQ ID NO: 140, SEQ ID NO: 160, SEQ ID NO: 180, SEQ ID NO: 200, SEQ ID NO: 220, and SEQ ID NO: 240.

19. The isolated nucleic acid of claim 17 or 18, wherein the contiguous nucleotide sequence is comprised of a sequence selected from SEQ ID Nos: 60-259.

20. The isolated nucleic acid of claim 17, 18, or 19, further comprising a complementary strand.

21. The isolated nucleic acid of claim 20, wherein the complementary strand is a separate annealed strand.

22. The isolated nucleic acid of claim 20, wherein the complementary strand is connected to the contiguous nucleotide sequence to form a self-annealed single strand.

23. The isolated nucleic acid of any of claims 17-20, wherein one or more nucleic acids are modified.

24. A method for treating or alleviating neurodegeneration in a subject comprising administering the isolated nucleic acid of any of claim 17-21 to the subject.

25. An isolated peptide comprising an amino acid sequence selected from the group consisting of SEQ ID NOS: 260-302.

26. A nucleotide encoding the isolated peptide of claim 25.

27. An antibody comprising complementary determination regions that bind the isolated peptide of claim 25.

28. The antibody of claim 27, wherein the antibody is a monoclonal antibody.

29. The antibody of claim 27, wherein the antibody is a polyclonal antibody.

30. A method of determining the onset and/or progression of Alzheimer's disease in a subject, comprising obtaining a sample from the subject and administering to the sample the antibody of claim 27.

31. The method of claim 30, further comprising initiating treatment upon determining when the antibody binds an antigen in the sample.

32. The method of claim 30, further comprising determining the concentration of antigen in the sample and obtaining a Braak score therefrom.

33. A method for measuring a Braak score in a subject comprising obtaining a sample from the subject; administering to the sample the antibody of claim 26; and, determining the concentration of antibody binding within the sample, wherein higher antibody binding increases the Braak score.
